(19) United States
(12) Patent Application Publication
(10) Pub. No.: US 2026/0283519 A1
(43) Pub. Date: Sep. 24, 2026

(54) STRETCHABLE, RECHARGEABLE, MULTIMODAL HYBRID ELECTRONICS FOR DECOUPLED SENSING TOWARD EMOTION DETECTION

(71) Applicant: The Penn State Research Foundation, Universty Park, PA (US)

(72) Inventors: Huanyu Cheng, University Park, PA (US); Libo Gao, Fujian (CN); Yangbo Yuan, University Park, PA (US)

(73) Assignee: The Penn State Research Foundation, University Park, PA (US)

(21) Appl. No.: 19/573,226

(22) Filed: Mar. 20, 2026

Related U.S. Application Data
(60) Provisional application No. 63/775,623, filed on Mar. 21, 2025.

(51) Int. Cl. A61B 5/16; A61B 5/00; A61B 5/01; A61B 5/024; A61B 5/1455

(52) U.S. Cl. CPC A61B 5/165; A61B 5/01; A61B 5/02416; A61B 5/14551; A61B 5/4266; A61B 2562/04; A61B 2562/166

(57) ABSTRACT

A stretchable, rechargeable, multimodal sensing device configured for emotion detection and psychological assessment. The device includes a flexible printed circuit board including a microcontroller unit, a non-transitory computer readable medium, a Bluetooth transceiver unit, and a sensor array. The sensor array includes a biaxial strain sensor configured to collect skin strain data, a temperature sensor configured to collect skin or environmental temperature data, a humidity sensor configured to collect skin moisture data, and a photoplethysmography sensor configured to collect heart rate data and blood oxygen saturation data. The collected sensor data may be transmitted wirelessly to external devices or cloud platforms for analysis, enabling personalized emotion recognition and mental health monitoring, among other applications.

### CROSS-REFERENCE TO RELATED APPLICATIONS

This application claims priority to U.S. Provisional Patent Application No. 63/775,623, which was filed on Mar. 21, 2025. The entirety of this application is incorporated by reference herein.

### STATEMENT REGARDING FEDERALLY SPONSORED RESEARCH DEVELOPMENT

This invention was made with government support under Grant Nos. 231939, DGE2243979 and ECCS2309323 awarded by the National Science Foundation and under Grant Nos. DA056242 and EB030140 awarded by the National Institutes of Health. The Government has certain rights in the invention.

### FIELD

The present disclosure generally relates to flexible bioelectronic sensing devices and systems for multimodal physiological and facial signal monitoring to enable emotion recognition and health assessment.

### BACKGROUND

With the collective advantages of soft, flexible/stretchable sensors and commercial rigid yet miniaturized high-performance device components, flexible hybrid electronics can provide a fully integrated device system for health monitoring, human-machine interfaces, and robotics. Multimodal sensing is crucial to characterizing physiological and psychological well-being in health monitoring. Given the rising stress levels in modern society, real-time emotion monitoring has become increasingly critical for early detection and timely intervention of psychological issues such as depression, anxiety, or panic attacks. While facial expressions can provide some insights into underlying emotions, the metrics that solely rely on them often lead to inaccurate classification and detection, as many individuals may not visibly express their feelings due to individual differences and social factors. Therefore, it is essential to combine facial expression analysis with other physiological parameters for a comprehensive understanding and improved prediction accuracy.

Valuable insights into emotional states can be obtained from changes in skin temperature, humidity, heart rate, and SpO₂ levels. For instance, skin temperature increases when an individual experiences surprise or anger, while it decreases during happiness, fear, or sadness. By primarily reflecting sweating levels, skin humidity rises during fear due to perspiration. Heart rate typically increases in response to emotions such as happiness, surprise, fear, or anger, while sadness often results in a decrease. For disgust, the temperature, humidity, and heart rate response may vary depending on the specific type of aversion experienced. Meanwhile, monitoring SpO₂ levels is valuable, as emotions that trigger panic attacks may lead to symptoms like dyspnea associated with a drop in SpO₂ levels. Therefore, more accurate emotion recognition can be possible with multi-dimensional data that integrate the physiological signals with facial expressions.

Although flexible sensors have been extensively reported for real-time tracking of strain, temperature, humidity, heart rate, and blood oxygen, it is still challenging to simultaneously detect these signals with high accuracy without crosstalk. Efforts to minimize coupling have started with intuitive strategies. For instance, stretchable structures such as serpentine shapes commonly used for enhanced stretchability can naturally mitigate the strain effect. Combining materials with positive and negative temperature coefficients achieves temperature insensitivity, while breathable or encapsulated sensors reduce sensitivity to humidity. On the other hand, porous ionic membrane-based humidity sensors exhibit ion migration stability within a certain temperature range, and porous structure resists significant deformation under pressure. Therefore, it can maintain stable performance across a wide range of temperatures and pressures. Despite these advancements, existing research has mostly focused on individual sensors to measure two input signals. Thus, there is an urgent need to exploit advanced sensors that can effectively decouple more than two signals for precise and reliable sensing toward emotion recognition.

### SUMMARY

We have developed a stretchable, rechargeable, multimodal bioelectric device designed for decoupled sensing of multiple physiological and environmental signals. The device may be particularly useful in emotion detection and monitoring, where accurate and independent measurement of various input signals is critical for reliable analysis.

In particular, the device is configured to independently measure parameters such as biaxial strain, temperature, humidity, heart rate, and blood oxygen saturation (SpO₂), with minimized cross-interference. By employing optimized structural designs, the sensors maintain high sensitivity and accuracy under mechanical deformation. For example, the device may utilize a multilayered flexible printed circuit board (FPCB) and a vertically stacked sensor array, thus reducing overall device footprint and enhancing wearability for continuous, real-time monitoring.

Furthermore, the device incorporates wireless power and data transmission modules, enabling seamless integration with mobile devices and/or cloud platforms. This system can facilitate remote health monitoring and emotion detection through advanced neural network models.

In an exemplary embodiment, a sensing device includes a flexible printed circuit board including a microcontroller unit, a non-transitory computer-readable medium connected to the microcontroller unit, a Bluetooth transceiver unit connected to the microcontroller, and a sensor array connected to the microcontroller. The sensor array includes a biaxial strain sensor configured to collect skin strain data, a temperature sensor configured to collect skin or environmental temperature data, a humidity sensor configured to collect skin moisture data, and a photoplethysmography sensor configured to collect heart rate data and blood oxygen saturation data.

In some embodiments, the sensing device includes an inside surface configured to contact a user when in use, and an outside surface opposite the inside surface, and the sensor array is vertically stacked.

In some embodiments, the sensor array is vertically stacked such that the humidity sensor is proximal to the inner surface.

In some embodiments, the humidity sensor includes a hydrophobic layer with an opening to enable the humidity sensor to be exposed while protecting other sensors from moisture.

In some embodiments, the temperature sensor includes a hydrophobic layer to protect the temperature sensor from moisture.

In some embodiments, a rigid layer is positioned adjacent to the humidity sensor to minimize the effects of strain.

In some embodiments, a rigid layer is positioned adjacent to the temperature sensor to minimize the effects of strain.

In some embodiments, the sensor array is positioned such that the collected skin strain data, skin or environmental temperature data, skin moisture data, heart rate data, and blood oxygen saturation data are decoupled.

In some embodiments, the device exhibits interference-resistant performance, with a normalized relative resistance change (ΔR/R₀) of 2% or less over an x-strain range of 20% or less, a y-strain range of 20% or less, a temperature range of 30-40° C., and a relative humidity range of 30-80%.

In some embodiments, the biaxial strain sensor includes an x-axis strain sensor and a y-axis strain sensor.

In some embodiments, at least one of the biaxial strain sensor, the temperature sensor, the humidity sensor, and the photoplethysmography sensor are designed in a serpentine shape.

In some embodiments, each of the biaxial strain sensor, the temperature sensor, and the humidity sensor are designed in a serpentine shape.

In some embodiments, the device further includes a power source configured to facilitate wireless charging and recharging of the sensing device.

In some embodiments, the power source includes one or more charging coils designed in a serpentine shape.

In an exemplary embodiment, a method for detecting emotions includes providing the sensing device described above; collecting sensor data via the sensor array; and transmitting the collected sensor data to a machine learning model.

In some embodiments, the method further includes processing the collected sensor data via the machine learning model to detect emotions.

In some embodiments, the machine learning model is hosted on an input/output device or a server.

Further features, aspects, objects, advantages, and possible applications of the present disclosure will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present innovation will be more apparent from the following more particular description thereof, presented in conjunction with the following drawings. Like reference numbers used in the drawings may identify like components.

FIG. 1 is a schematic illustration of an exploded view of an exemplary multilayered sensing device.

FIG. 2 is a schematic illustration of an exploded view of sensors and other components of the exemplary multilayered sensing device shown in FIG. 1.

FIG. 3 is a schematic block diagram of circuit board components for an exemplary embodiment of the device.

FIG. 4 is a schematic illustration of an exemplary fabrication process of the sensor array, including a humidity sensor, a temperature sensor, and a strain sensor.

FIG. 5 is a schematic block diagram illustrating an exemplary embodiment of a system for transmitting sensor data collected by a device.

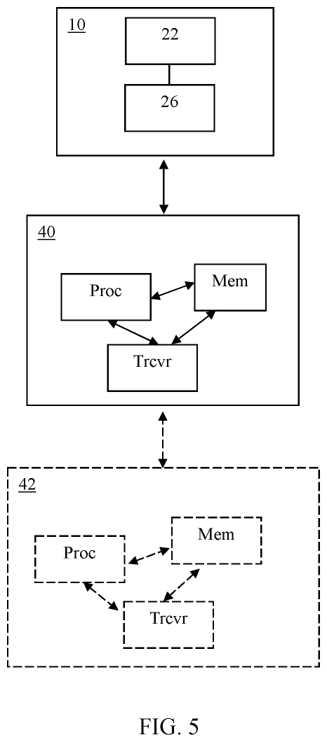

FIG. 6 shows optical images of exemplary (left) strain, (middle) temperature, and (right) humidity sensors.

FIG. 7 shows an optical image of an integrated sensing device (left) before and after (middle) bending or (right) twisting.

FIG. 8 shows an optical image of a flexible wireless powering and transferring circuit with an integrated PPG sensor upon bending between two fingers.

FIG. 9 shows the comparison between finite element analyses (FEA, top) and photographs (bottom) of an exemplary device under mechanical deformations, such as 20% stretching (left), bending of 150° (middle), and twisting of 60° (right).

FIG. 10 is a schematic of exemplary device components and data flow.

FIG. 11 shows an exemplary printed circuit board (PCB).

FIG. 12 shows (top left) a schematic diagram, (top right) optical images of a wireless charging coil, and the corresponding (bottom) FEA results under stretching, bending, and twisting.

FIG. 13 shows an optical image of an integrated device system before encapsulation with Ecoflex.

FIG. 14 is a schematic diagram showing an encapsulation process, along with the optical image of the model used for encapsulation.

FIG. 15 shows optical images of a sensing device: (left) front and (right) back sides.

FIG. 16 shows (left) a photograph of the setup for wireless charging and (right) the measured output voltage signal from the receiving coil.

FIG. 17 is a diagram of potential applications of the device combined with neural network analysis for emotion monitoring and analysis.

FIG. 18 includes graphs demonstrating normalized relative changes of the X-axis strain sensor as a function of (left) x-axis tensile strain, (middle) temperature, and (right) humidity.

FIG. 19 includes graphs demonstrating normalized relative changes of the Y-axis strain sensor as a function of (left) y-axis tensile strain, (middle) temperature, and (right) humidity.

FIG. 20 includes graphs showing the response and recovery time of the strain sensor in the (left) X- and (right) Y-directions.

FIG. 21 includes graphs showing the dynamic response of the strain sensor in the (left) X- and (right) Y-directions.

FIG. 22 includes graphs showing the limit of the detection of the strain sensor in the (left) X- and (right) Y-directions.

FIG. 23 includes graphs showing hysteresis characteristics of the strain sensor when stretched by (left) 25% along the X-axis and (right) 50% along the Y-axis.

FIG. 24 includes plots showing long-term durability test of the strain sensor over (top) 35% stretching in the X-direction and (bottom) 80% stretching in the Y-direction.

FIG. 25 shows an equivalent circuit diagram of the strain sensors, where Rx and Ry represent the strain sensors along the X- and Y-directions, R1 and R2 are voltage divider resistors, and A0 and A1 are signal reading ports.

FIG. 26 includes graphs demonstrating normalized relative changes of the temperature sensor as a function of (left) temperature, (middle) humidity, and (right) strain.

FIG. 27 includes graphs demonstrating normalized relative changes of the humidity sensor as a function of (left) humidity, (middle) temperature, and (right) strain.

FIG. 28 is a graph showing the response/recovery time of the temperature sensor.

FIG. 29 is a graph showing the repeatability response of the temperature sensor.

FIG. 30 is a graph showing the response/recovery time of the humidity sensor.

FIG. 31 is a graph showing the repeatability response of the humidity sensor.

FIG. 32 includes graphs showing the normalized relative resistance changes of (top) X-axis strain sensor and (bottom) Y-axis strain sensor with the applied strain along the sensing direction, temperature, and humidity all within the physiologically relevant ranges.

FIG. 33 includes graphs showing the normalized relative resistance changes of the (top) temperature sensor and (bottom) humidity sensor with the applied temperature, relative humidity, and stretching all within the physiologically relevant ranges.

FIG. 34 is a graph showing physiologically relevant ranges of strain, temperature, and relative humidity on the human skin.

FIG. 35 includes (left) an optical image, (middle) a schematic diagram, and (right) a cross-section scanning electron microscopy (SEM) image of an exemplary sensor.

FIG. 36 shows a temperature distribution of a sensor in an environment of 300 K.

FIG. 37 shows a humidity distribution of a sensor in an environment with a water vapor density of 0.7626 kg/m³, and an SEM and porosity analysis image of carbon nanotubes.

FIG. 38 shows the strain distribution in a serpentine electrode (left) without PET strain isolation under 20% X-axis stretching and (middle) with PET strain isolation under equivalent tensile stress, along with (right) the SEM image of the serpentine electrode (without cracks due to PET).

FIG. 39 shows the strain distribution in the serpentine electrode of the sensors (left) without and (right) with PET strain isolation under a tensile strain of 20% along the Y-direction.

FIG. 40 shows the strain distribution of a strain sensor upon stretching along the (top left) X- and (bottom left) Y-directions, with (right) SEM images showing the evolution of the crack pattern with the tensile strain: 5% (left), 40% (middle), and 80% (right).

FIG. 41 shows an optical image of an integrated sensing device placed on the face of the human subject.

FIG. 42 is a schematic flow diagram of an exemplary machine learning algorithm for training and classifying different facial expressions.

FIG. 43 includes graphs showing (left) loss and (right) accuracy rates of training and validation sets over 100 iterations/epochs of a training process.

FIG. 44 shows a confusion matrix of emotion recognition after 100 iterations.

FIG. 45 includes graphs showing representative strain measurements along the (left) X- and (right) Y-directions for six facial expressions, labeled 1 to 6 for happiness, surprise, fear, sadness, anger, and disgust.

FIG. 46 shows a K-means clustering plot corresponding to FIG. 45.

FIG. 47 shows K-means clustering plots of six facial expressions (i.e., fear, sadness, surprise, disgust, anger, and happiness) from three different human subjects: (left) #1 (male, age: 23, weight: 70 kg, height: 175 cm), (middle) #2 (male, age: 23, weight: 62 kg, height: 168 cm), and (right) #3 (female, age: 30, weight: 55 kg, height: 155 cm).

FIG. 48 is a plot showing a comparison of the predicted accuracy of six facial expression features from the three human subjects.

FIG. 49 includes graphs showing response variations of the (left) temperature and (right) humidity sensors in resting/moving states.

FIG. 50 includes graphs showing response variations of the (left) temperature and (right) humidity sensors under different emotions.

FIG. 51 includes graphs showing changes in the sensor responses to the variations of the subject's emotion at different times of the day: (top) morning, (middle) afternoon, and (bottom) evening.

FIG. 52 shows photographs of an integrated hybrid device attached to different body parts.

FIG. 53 includes graphs showing a comparison of the measured (left) temperature and (right) humidity from different body locations: (1) face, (2) forehead, (3) arm, and (4) fingertip.

FIG. 54 includes graphs showing raw normalized photoplethysmogram (PPG) voltage outputs collected from the photodetector for (left) infrared and (right) red light-emitting diodes (LEDs) for calculating the heart rate and SpO₂ signals.

FIG. 55 is a graph showing robust heart rate and SpO₂ measurements from the PPG signal in the presence of approximately 10% cyclic stretching and release on the forearm.

FIG. 56 is a plot showing (top) SpO₂ data obtained from different body parts in resting and intermittent breath-holding states, (middle) changes in the heart rate obtained from the face in resting/moving states, and (bottom) under different emotional states.

FIG. 57 shows exemplary (left) web and (right) mobile-based cloud monitoring interfaces.

### DETAILED DESCRIPTION

The following description is of exemplary embodiments and methods of use that are presently contemplated for carrying out the present invention. This description is not to be taken in a limiting sense but is made merely for the purpose of describing the general principles and features of various aspects of the present invention. The scope of the present invention is not limited by this description.

Embodiments generally relate to a stretchable, rechargeable, multimodal bioelectric sensing device capable of continuous physiological and environmental signal monitoring for emotion detection. The device may include a combination of sensors, flexible circuitry, and other components configured to conform to various parts of a user's body. The sensors are generally designed to independently detect multiple physiological signals relevant to emotional state assessment, such as biaxial strain, skin temperature, skin humidity, heart rate, and blood oxygen saturation (SpO2). These sensors may be constructed to support stretchability, mechanical durability, and decoupled operation. As will be described in further detail below, the sensors may be arranged in either a single-layer or a multilayer (e.g., bi-layered) configuration, depending on design preferences and application requirements.

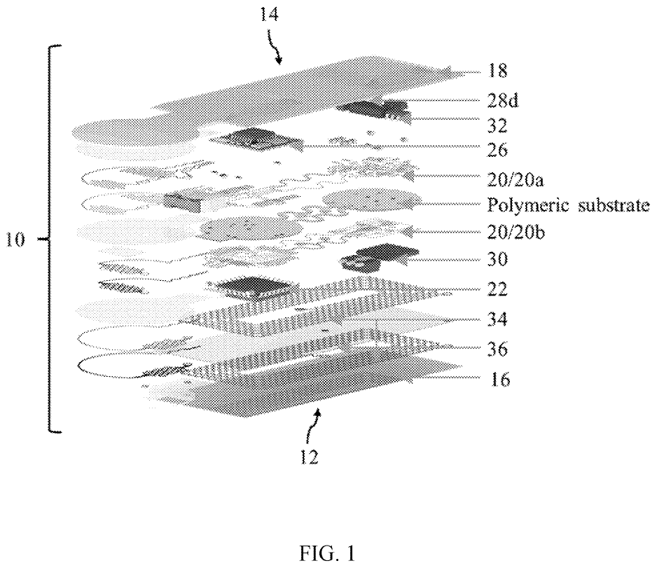

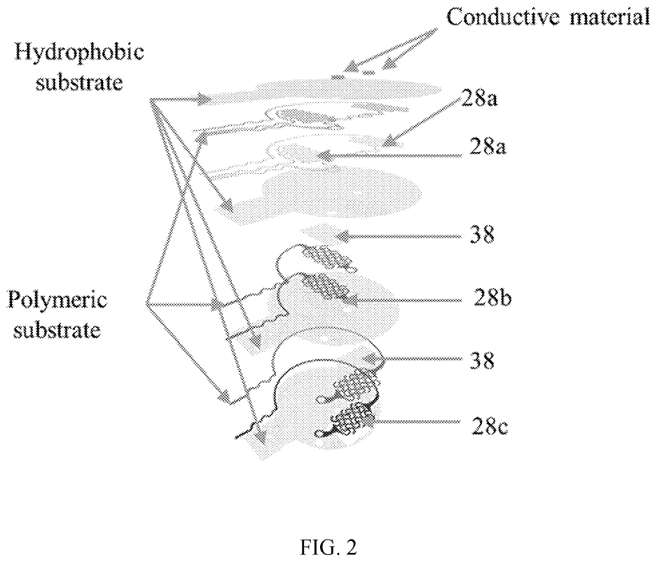

Referring to FIGS. 1 and 2, a device 10 has an inside surface 12 and an outside surface 14. In use, the inside surface 12 is configured to contact the user's skin, while the outside surface 14 is exposed and visible to the user. The inside surface 12 may be formed by a first encapsulation layer 16, and the outside surface 14 may be formed by a second encapsulation layer 18, such that these layers join to define an encapsulated region. The term “encapsulated region” generally refers to an area that is enclosed or surrounded on all sides, preventing exposure to external surroundings.

The first and second encapsulation layers 16, 18 may be formed from any suitable flexible material. These materials are typically chosen for their ability to accommodate mechanical deformations such as stretching, bending, and twisting. They may also be waterproof or water resistant, allowing the device 10 to be worn safely during wet environmental conditions, aquatic activities, or when the user is sweating. In some embodiments, both layers may be constructed from skin-compatible elastomeric materials, such as Ecoflex. Ultimately, the device 10 may be designed for conformal contact with the skin, enabling continuous, real-time monitoring of physiological and facial signals.

The device 10 may further include an adhesive or skin-contact layer on the first encapsulation layer 16, enabling the device to be removably attached to the user's skin.

The device 10 includes one or more flexible printed circuit boards 20 positioned within the encapsulated region. These circuit boards may be printed on flexible polymeric substrates, allowing them to bend and stretch in concert with the device 10. The flexibility of the circuit boards supports mechanical deformation without compromising electrical integrity.

The circuit boards 20 include a plurality of electrical components, such as a microcontroller unit (MCU) 22, which may be connected to a non-transitory memory 24 or other computer-readable medium, a Bluetooth transceiver unit 26, and a sensor array 28 including a plurality of sensors.

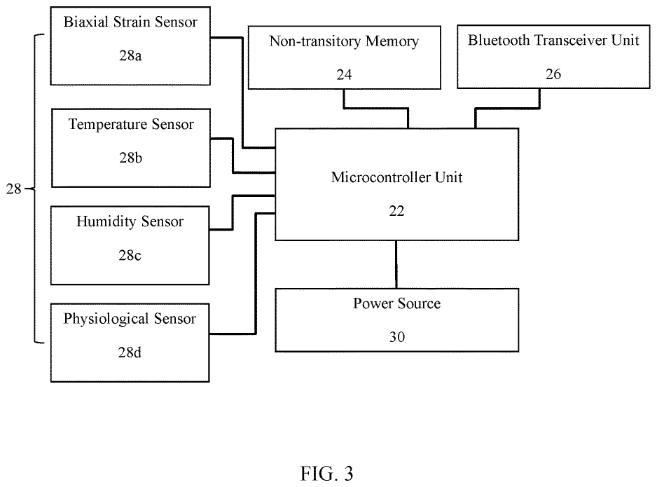

As seen in FIG. 3, the microcontroller unit 22 interfaces with the other components of the circuit boards 20. A power source 30 can also be positioned within or in contact with the circuit boards 20 to supply power to the device's electronics.

The power source 30 generally provides the primary voltage supply for the device 10 and may be a rechargeable battery, such as a lithium-ion battery. The power source 30 may include a charging module 32, which facilitates wireless recharging.

In certain embodiments, the power source 30 may incorporate one or more charging coils 34, designed with a serpentine shape to enhance stretchability and to naturally mitigate strain effects during device deformation. One or more spacers 36 (e.g., made of PET) may be positioned to separate the coils 34 from each other or from the circuit boards 20.

To charge the device 10, it may be positioned near or on an external charging device, initiating the wireless charging process. The AC voltage received by the coils 34 may be converted into a DC voltage via a rectifier and filter circuitry, which then recharges the power source 30. This configuration supports continuous, real-time monitoring over extended periods.

The placement of the power source 30, charging module 32, and/or charging coils 34, is not particularly limited. In some embodiments, the charging module 32 and/or the coils 34 may be placed in proximity to the outside surface 14 of the device 10, allowing for efficient wireless charging when the device is placed on or near an external charging station, and advantageously preserving adhesion.

The device 10 may include a power switch or activation mechanism, such as a button or automatic contact detection, to control power cycling.

The sensor array 28, included on the circuit boards 20, encompasses a plurality of sensors configured to collect physiological and environmental data for emotion detection and health assessment. The sensors may be communicatively connected to the microcontroller unit 22 via interfaces such as SPI, I²C, UART, or similar protocols, and may be implemented as integrated chips within a system-on-chip (SoC) architecture to support a small form factor.

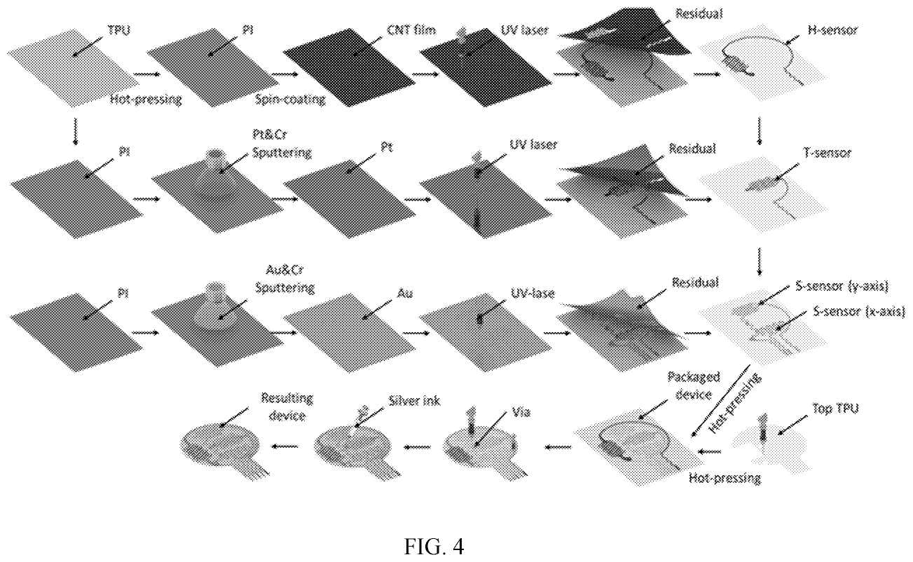

The sensor array 28 may include:
(i) Biaxial strain sensors 28a: These sensors are configured to detect deformation of the user's skin or muscles in two orthogonal directions, namely, the X-axis and Y-axis, allowing for comprehensive capture of multidirectional movements associated with facial expressions or skin stretch. The sensors may be patterned with serpentine geometries to enhance stretchability and mechanical resilience. The X-axis sensor may be sensitive primarily to lateral deformation, while the Y-axis sensor responds to vertical deformation. The biaxial sensors 28a may be constructed by sputtering conductive metals, such as silver, gold, or other electrically conductive metals, onto flexible substrates, followed by laser patterning and peeling (see FIG. 4). Their electrical responses may be minimally affected by temperature, humidity, or strain over broad ranges, ensuring high measurement accuracy.

The position of the biaxial strain sensors 28a on the circuit boards 20 and within the encapsulated region is not particularly limited.
(ii) Temperature sensors 28b: These sensors are configured to measure skin or environmental temperature, which can fluctuate during emotional states such as surprise, anger, happiness, or fear. These sensors may be formed by sputtering a thin film of a noble metal like platinum or other temperature-sensitive conductive materials onto a flexible substrate, patterned into serpentine shapes to support stretchability (see FIG. 4). In some embodiments, the sensors incorporate a hydrophobic layer to protect the sensors from moisture. In certain embodiments, a spacer or isolation layer 38 (e.g., made of PET) may be placed adjacent to the sensors to further minimize the effects of strain.

The sensors are calibrated over a range of approximately 20° C. to 90° C., with their electrical resistance changing predictably with temperature. The position of the biaxial strain sensors 28a on the circuit boards 20 and within the encapsulated region is not particularly limited.
(iii) Humidity sensors 28c: These sensors are designed to detect skin moisture and perspiration, which may increase during emotional arousal or stress. These sensors are often constructed from porous, ion-conductive materials such as carbon nanotube (CNT) deposited onto a flexible substrate (see FIG. 4). The sensing element is patterned into serpentine geometries to ensure stretchability and to reduce sensitivity to strain. In some embodiments, the sensors incorporate a hydrophobic layer with an opening that allows the humidity sensing region to be exposed to ambient air while protecting other electrical components from moisture. In certain embodiments, a spacer or isolation layer 38 (e.g., made of PET) may be placed adjacent to the sensors to further minimize the effects of strain.

The humidity sensor 28c generally exhibits high sensitivity (e.g., approximately 0.2472% RH), within a linear range from around 30% to 80% relative humidity.

The humidity sensor 28c may be positioned close to the skin surface, often proximal to the inside surface 12 of the device 10, with exposed areas or apertures in the inside surface 12 allowing direct contact with skin moisture for accurate measurement.
(iv) Physiological sensor 28d: These sensors may include a photoplethysmography (PPG) sensor with embedded LEDs and photodetectors configured to non-invasively measure blood volume changes in superficial vasculature. The PPG sensors generate pulsatile signals that can be processed to derive heart rate and blood oxygen saturation (SpO2). Additional sensors, such as electrocardiogram (ECG) or inertial measurement unit (IMU) sensors, may be incorporated into the device 10.

The placement of the physiological sensors 28d is flexible within the device 10, but may be positioned to maximize contact and signal quality.

In one embodiment, the device 10, and in particular the circuit boards 20, may be arranged in a vertically stacked, multilayer configuration to optimize signal integrity, reduce interference, and support a compact form factor. This multilayer architecture may include a first circuit layer 20a positioned closer to the outside surface 14 of the device, and a second circuit layer 20b positioned nearer to the inside surface 12.

In this configuration, the sensor array 28 and other electronic components of the device 10 may be organized into a vertically stacked multilayer assembly. Such an arrangement facilitates effective decoupling of signals, minimizes cross-interference between sensors and circuits, and allows for a smaller overall device size. The layers may be bonded together via hot pressing, which ensures a secure mechanical connection, and interconnected through laser-cut vias filled with a conductive material such as silver or gold paste. This approach supports high-density integration, reliable electrical connections, and mechanical robustness under deformation.

In a specific embodiment, the first circuit 20a may include components such as the Bluetooth transceiver unit 26, the wireless charging module 32, biaxial strain sensors 28a, and physiological sensors 28d. Conversely, the second circuit 20b may comprise the microcontroller unit 22, the power source 30, temperature sensors 28b, and humidity sensors 28c. It is understood, however, that this configuration is merely illustrative; the placement of components across the layers can be optimized or rearranged based on design considerations, manufacturing constraints, or application-specific requirements.

In an alternative embodiment, the device 10 may be implemented as a single-layer configuration, where sensors and electronic components are distributed spatially across the surface of the device. In this embodiment, sensors may be physically separated and electrically isolated to minimize coupling effects, potentially simplifying fabrication and assembly processes. While this flat configuration may be less compact than the multilayer arrangement, it can be advantageous in applications where ease of manufacturing or accessibility outweigh the benefits of miniaturization.

Following data collection, the sensor signals may be converted into voltage variations, amplified, and digitized via analog-to-digital converters (ADCs) integrated within the circuit boards 20. As will be described in further detail, the processed data can then be transmitted wirelessly, such as via Bluetooth, to an external input/output device 40 or a server 42 for further analysis, evaluation, and/or storage.

The design of the device 10 ensures that sensor data are collected with high fidelity and with minimal cross-interference, which can be crucial for accurate emotion and physiological state assessment. This robustness enables reliable monitoring of subtle changes in physiological parameters, such as skin temperature, humidity, heart rate, and SpO₂, that are associated with emotional states.

The device 10 is capable of monitoring physiological signals in real-time, providing important insights into emotional and psychological conditions such as anxiety, stress, and panic disorders.

The device 10 is designed for versatile placement on various parts of a user's body, depending on the specific application and the type of physiological or environmental signals to be monitored. It may be attached to regions such as the face (e.g., cheeks, forehead, chin), forehead, wrist, arm, fingertip, or other skin surfaces where relevant physiological signals are accessible. The flexible and conformal nature of the device 10 allows it to adhere securely and comfortably to these regions during movement and daily activities.

The device 10 is particularly suited for applications including, but not limited to, mental health monitoring, stress and anxiety assessment, emotion recognition, human-computer interaction (HCl), and telemedicine. It can facilitate remote psychological evaluation, real-time stress management, and personalized health tracking. The data collected can be transmitted wirelessly to smartphones, tablets, or cloud platforms, enabling continuous monitoring and analysis over extended periods.

The device can also be employed in research environments to study physiological responses to various stimuli, such as emotional stimuli, physical activity, or environmental changes. Its ability to decouple multiple physiological signals and integrate facial expression analysis allows for a comprehensive assessment of an individual's emotional and physiological state, providing valuable insights into human behavior, mental health, and neurological conditions.

As stated above, the data collected by the sensor array 28 can be transmitted to an input/output device 40 (e.g., a smartphone, tablet, laptop computer, personal computer, etc.). For example, the collected data may be transmitted using the Bluetooth transceiver unit 26 (see FIG.). The Bluetooth transceiver unit 26 can be configured as a wireless interface for the transmission of sensor data via the device's wireless connection with an input/output device 40. For example, the Bluetooth transceiver unit 26 can be a Bluetooth Low Energy (BLE) system-on-chip coupled to the microcontroller 22 to facilitate Bluetooth wireless communications with the input/output device 40. The Bluetooth transceiver unit 26 can also have an antenna that is connected to the microcontroller 22 and helps facilitate the transmission of sensor data.

In some embodiments, the data collected by the sensor array 28 may be continuously streamed to the input/output device 40. In other embodiments, the data collected by the sensor array 28 may be periodically streamed to the input/output device 40 (e.g., non-continuously at pre-determined intervals). The data collected by the sensor array 28 can also be stored within the non-transitory computer-readable medium for an extended period of time before being transmitted to the input/output device 40.

It should be appreciated that the input/output device 40 is a computer device that has at least one processor (Proc) connected to at least one non-transitory computer-readable medium (e.g., memory, a hard drive, etc.) (Mem) and at least one transceiver unit (e.g., a network transceiver, etc.) (Trcvr).

If a device 10 is connected to the input/output device 40, the sensor data collected by the sensor array 28 can be sent to the device. The data being transmitted to the input/output device 40 may be stored data or a continuous stream of current data. The data may be collected continuously or at pre-selected monitoring intervals (e.g., every five seconds, every minute, etc.). Each sensor of the sensor array 28 may collect data continuously or discretely within pre-selected sensor measurement intervals spaced apart from each other by a pre-selected monitoring interval time period.

In an exemplary embodiment, the sensor data can be obtained from sensors of the sensory array 28 via at least one sensor interface that connects the sensors to the microcontroller unit 22. The sensor data can be processed via the sensor interface. For example, the sensor data may be packaged as a data packet. The processed data can then be transmitted for secure storage and/or stored on the computer-readable medium of the device 10. When the stored sensor data is transmitted to the input/output device 40, transmission of related data can be included with the transmitted sensor data to identify the transmission process used to transmit the sensor data, time of transmission, time at which sensor data was stored and/or collected, sensor device identifier for each type of sensor data, as well as other data relevant to the collection and storage of the sensor data for subsequent evaluation by the input/output device 40 and/or other computer device.

In some embodiments, the data may alternatively or subsequently be sent to a server 42 that can be connected to the input/output device 40 (e.g., via a network connection and application programming interface (API) the server may have with the input/output device 40 running the application, etc.). The server 42 may be configured to store the sensor data of the device 10. The forwarding of such data can be provided via a communication connection that the device 10 directly has with an input/output device 40 such that data is sent to the server 42 via the input/output device 40.

It should be appreciated that the server 42 can be configured as a computer device that has at least one processor (Proc) connected to at least one non-transitory computer-readable medium (e.g., memory, a hard drive, etc.) (Mem) and at least one transceiver unit (e.g., a network transceiver, etc.) (Trcvr). The server 42 can be configured as a database server, for example. The server 42 can also be configured as a host server that hosts services provided to the user of the device 10 via communications exchanged between the server 42 and the device 10, and/or at least one input/output device 40.

Once the data is transmitted to the input/output device 40 and/or the server 42, the data may be analyzed and evaluated. For example, the data can be processed using artificial intelligence or machine learning algorithms stored on the input/output device 40 and/or the server 42. In particular, the input/output device 40 and/or server 42 may run a program that uses the collected sensor data along with a model trained via a machine learning process that received the collected sensor data and processes that data to determine an output (e.g., emotion detection, monitoring, recognition, etc.).

For example, a machine learning model may use data together with machine learning algorithms that undergo a training process to prepare a trained model. In this case, the model may use a predetermined training dataset and machine learning algorithms to prepare a trained model that may be utilized to process collected data to generate an output (e.g., emotion detection, monitoring, recognition, etc.).

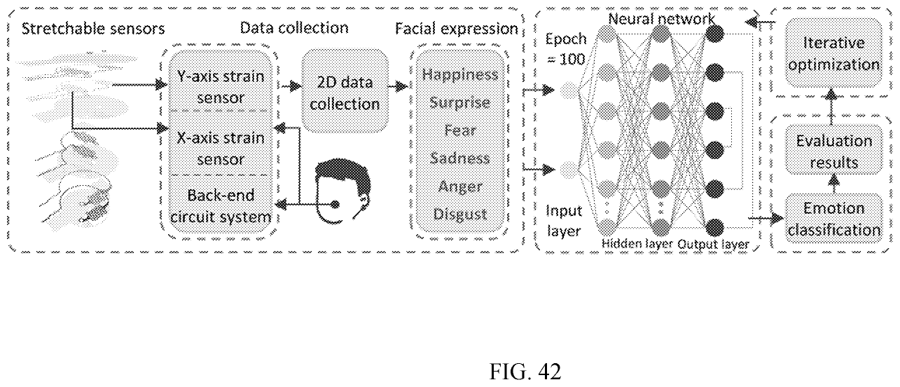

The framework (see FIG. 42) of the model employs a specialized deep learning architecture designed to provide reliable emotion detection and assessment resilient to strain.

In particular, a neural network model may be employed within the system to enable high-accuracy emotion recognition and precise facial expression classification. The neural network can effectively differentiate facial expressions, such as happiness, surprise, fear, sadness, anger, and disgust, using strain data that is independent of temperature and humidity, thereby reducing the influence of environmental and physiological variations. The model processes time-sequence physiological signals collected by the sensors, leveraging the distinct patterns associated with each emotional state. Its architecture may include hidden layers that extract complex features from the input data and an output layer that categorizes the facial expression. Since each emotion correlates with a specific set of physiological changes over time, the data processed by the neural network facilitates reliable recognition of facial expressions.

The input/output device 40 and/or the server 42 may comprise a display unit and/or a graphical user interface (GUI) for providing information to the user regarding collected sensor data. For example, the device 10 may transmit data collected from the sensor array 28 to the input/output device 40 and/or the server 42, and the collected data may be displayed in the form of a graph, chart, text, or other display via the GUI. The GUI may further display outputs or other collected data to the user.

### EXAMPLES

### Methods

Fabrication of the Temperature, Humidity, and Strain Sensors: The structural design of the sensors and the wireless charging coil was performed using Autodesk CAD 2022. The fabrication process for all sensors began by bonding a 30 μm-thick PI film to a 120μm-thick TPU film through hot pressing at 145° C. for 90 seconds. After plasma treatment, specific sensing materials were deposited onto the PI film via magnetron sputtering: Cr/Pt for the temperature sensor (160 s for Cr and 600 s for Pt at 40 mA, with a vacuum pressure of 18 Pa and 14 Pa, respectively) and Cr/Au for the strain sensor (160 s for Cr and 300 s for Au, with a vacuum pressure of 18 Pa and 7 Pa, respectively), with Cr as the adhesive layer. As for the humidity sensor, a 5 mg/mL CNT solution was spin-coated on the plasma-treated PI. Patterning of the sensing material layer with a UV laser (25 kHz, 7 cycles) on a laser suction table into the designed shapes, followed by removing excess material using tweezers, completed the fabrication of the temperature, humidity, and strain sensors.

Assembly of the Sensor Device: After laser cutting the TPU encapsulation layer to expose the humidity sensor, different layers were stacked and hot-pressed at 145° C. for 150 seconds from top to bottom: TPU encapsulation, humidity sensor with a rigid PET isolation layer underneath, temperature sensor layer with a rigid PET isolation layer underneath, and strain sensor layer. Next, silver paste is injected into vias created at specified locations with laser cutting (50 kHz, 20 cycles). Placing the assembled sensing device on a heating plate at 90° C. for 3 hours cured the silver paste in the vias and completed the fabrication.

Fabrication of the Wireless Charging Coil: The fabrication of the wireless charging coil started with attaching a 30 μm-thick copper foil to a glass plate coated with a 400 μm-thick PDMS layer. Laser cutting (50 kHz, 5 cycles) created patterned Cu foil for use as the upper and lower coils, followed by transfer onto a water-soluble tape. After subsequently attaching these two coils to the top and bottom surfaces of TPU and hot-pressing at 145° C. for 90 s, submerging in deionized water for two minutes dissolved the water-soluble tape (removal facilitated by using a brush). After drying the surface, vias at the central connection points were created by laser cutting (50 kHz, 5 cycles) and filled with injected silver paste. Curing on a heating plate at 90° C. for 3 hours completed the fabrication of the wireless charging coil.

Design of the FPCB: The FPCB, consisting of several key modules for efficient operation, was designed using Altium Designer (21.0.4). The data acquisition module included the PPG (MAX30102) sensor for heart rate and SpO₂ monitoring, along with a voltage divider circuit to measure resistance changes in the strain, temperature, and humidity sensors. Data processing and wireless transmission were managed by a main control MCU (ATmega328P) and a Bluetooth module (CC2640R2F). A 5V rechargeable lithium battery was regulated by the HT7133 chip in the power supply module to maintain a stable voltage output of 3.3 V for the system. Additionally, the wireless charging module with diodes, capacitors, and an XKT-R03 chip converted the AC from the wireless charging coil into DC for recharging the battery.

Assembly and Encapsulation of the Device: After applying the soldering flux paste and soldering the connection points between the wireless charging coil and FPCB using low-temperature reflow, the fabricated sensors were connected to the FPCB via flexible printed circuit connectors to complete the device assembly. Next, a milling machine was used to create a mold from an aluminum plate. After spraying Easy Release on the mold surface, Eco-flex (A:B=1:1 mixed with white silicone dye) was poured into the mold and cured at 80° C. for 1 hour. Next, the assembled device was placed onto the cured silicone in the mold. Another layer of silicone poured over the device was cured at 80° C. for 1 hour. Finally, the encapsulated device removed from the mold was laser cut (50 kHz, 5 cycles) over the outline to prepare the device.

Structural, Mechanical, and Electromechanical Characterizations: The structural and morphological characterizations of the materials and sensors were performed using field emission scanning electron microscopy (Quanta 450). The porosity of the CNT network was calculated using ImageJ based on the SEM image. The mechanical properties of the strain sensor were evaluated using a universal testing machine (ZhiQu, 990B). The temperature sensor was tested on a heating table, whereas the measurements of the humidity sensor were carried out in a chamber with controlled humidity levels. For electromechanical characterizations, the resistance values were measured using an LCR impedance analyzer (Tonghui, TH 2827A) and an IM3536 LCR meter. Characterization experiments were conducted with at least four samples tested for each condition.

Applications of the Device: Machine learning algorithms were implemented using Python 3.11, with Replit as the development platform. Training data were collected from five human subjects (#1 (male, age: 25, weight: 66 kg, height: 179 cm), #2 (male, age: 25, weight: 70 kg, height: 181 cm), #3 (male, age: 24, weight: 60 kg, height: 177 cm), #4 (female, age: 23, weight: 55 kg, height: 165 cm), #5 (female, age: 23, weight: 54 kg, height: 163 cm)), with each repeating six facial expressions (i.e., happiness, surprise, fear, sadness, anger, and disgust) 100 times. The six facial expressions were repeated 50 times by three new participants (#1 (male, age: 23, weight: 70 kg, height: 175 cm), #2 (male, age: 23, weight: 62 kg, height: 168 cm), #3 (female, age: 30, weight: 55 kg, height: 155 cm)) served as the validation data. The mobile application for receiving and displaying the sensing data was developed using Java. Both the web-based and mobile-based cloud platforms were developed using Ali Cloud.

### Results

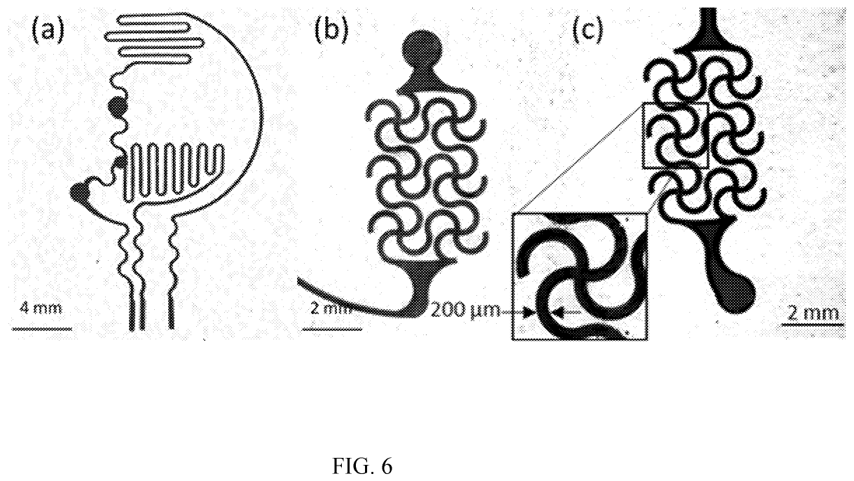

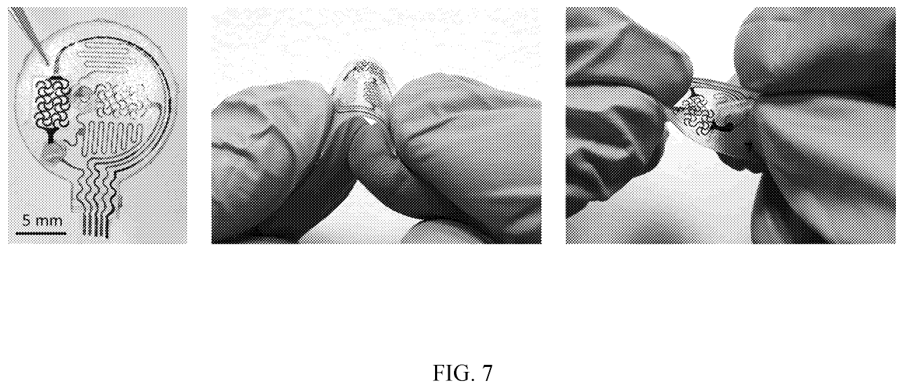

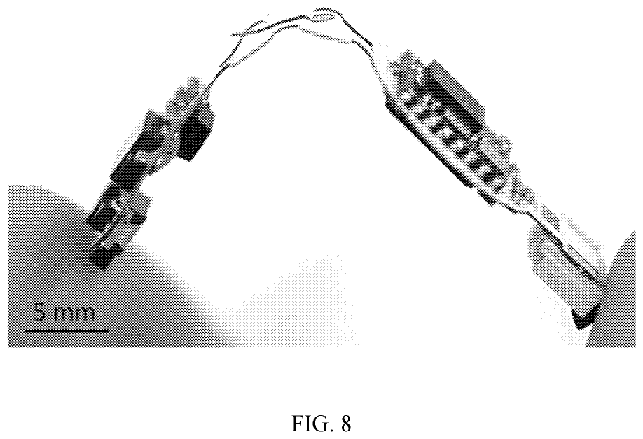

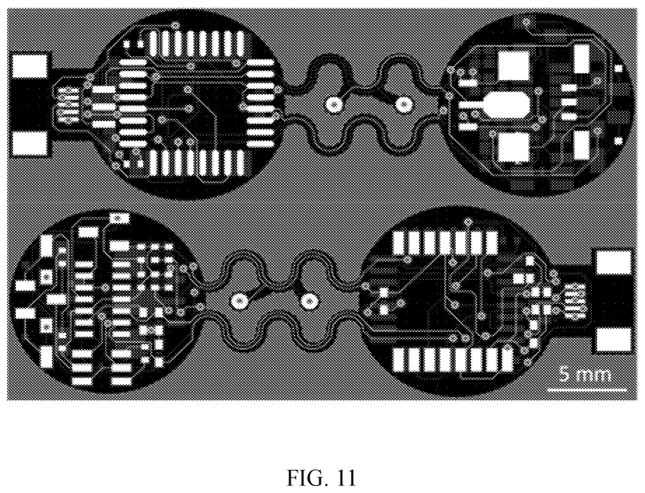

The rechargeable hybrid sensing device system featured a combination of in-house fabricated stretchable sensors with a bi-layered flexible printed circuit board (FPCB) and a wireless charging module all encapsulated by a silicone elastomer (Ecoflex) from both the top and bottom (FIG. 1). The stretchable sensors integrated temperature and humidity sensors with strain sensors along both the X- and Y-directions, designed to exhibit minimized interferences, for decoupled sensing of these signals at enhanced accuracy (FIG. 2). In brief, sensors were fabricated on the polyimide (PI) substrate bonded to thermoplastic polyurethane (TPU) using hot pressing (see the fabrication details in FIG. 4). The humidity sensor utilized the use of spin-coated carbon nanotube (CNT) solution, whereas the temperature and strain sensors were fabricated through magnetron sputtering of platinum (Pt) and gold (Au) with chromium (Cr) as an adhesive layer. The patterns of the sensors were facilely created by direct laser cutting and peeling (FIG. 6). The impact of strain on the temperature and humidity sensors was minimized by placing a relatively rigid polyethylene terephthalate (PET) spacer beneath them for strain isolation. Only the humidity sensor was exposed to the outside, where the hydrophobic TPU layer prevented the permeation of water molecules to the strain and temperature sensors. The sensors arranged in a multi-layer layout for minimized footprint were bonded through hot pressing, with interconnections formed by silver paste filled in the laser-cut via holes. The combination of soft elastomers and inorganic materials with stretchable structures allowed the resulting sensing device to mechanically deform upon stretching, bending, and twisting without damage (FIG. 7). To provide a facile connection for the sensors in the multi-layer layout, the FPCB (FIG. 8) on a PI layer also featured a bi-layered design, with Bluetooth, PPG sensor, and wireless charging modules on the upper circuit along with a microcontroller unit (MCU) and rechargeable lithium battery on the lower circuit (FIG. 11).

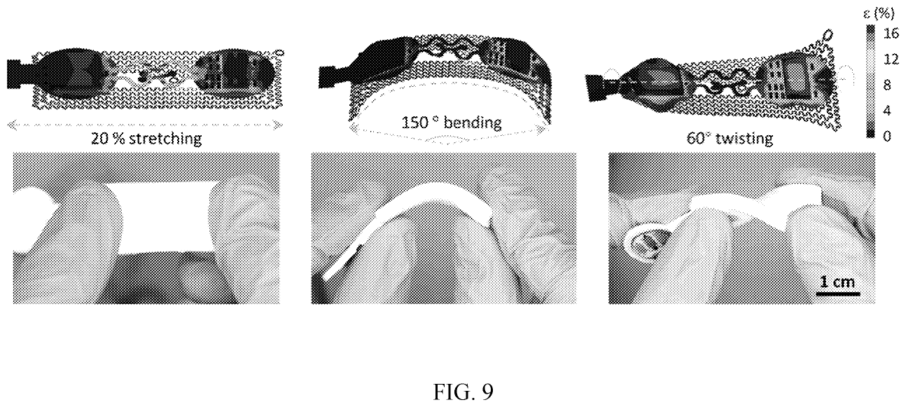

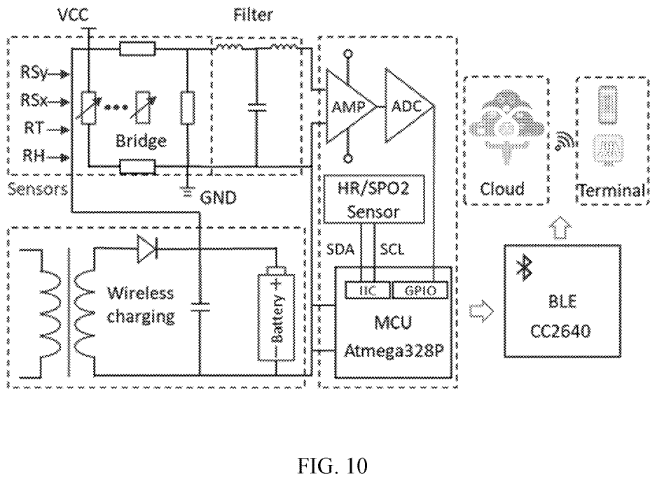

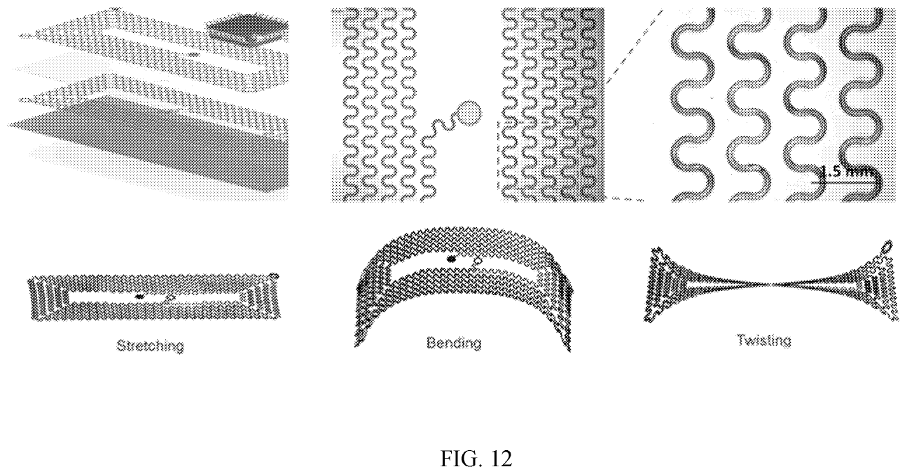

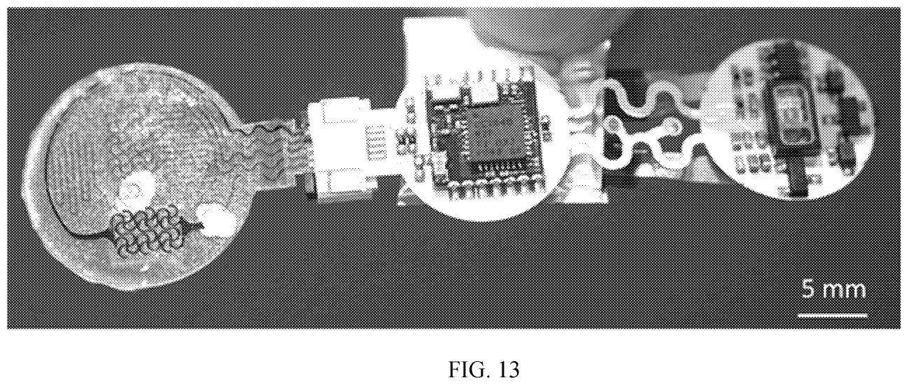

As motion artifact presents a persistent challenge for accurate detection of physiological signals from skin-interfaced sensors/devices, the integrated device sensing system was designed to robustly operate even in the presence of motions or movements. As confirmed by finite element analysis (FEA), the device components experienced negligibly small strains and exhibit robust performance upon 20% stretching, 150° bending, and 60° twisting (FIG. 9). The measured resistance changes from the temperature, humidity, and biaxial strain sensors were converted into voltage variations (FIG. 10). These signals were then filtered, amplified, and converted into digital data, before being transmitted to the MCU via four General-Purpose Input/Output ports for detection. The PPG sensor sent data to the MCU through the Serial Clock Line and Serial Data Line connections. After processing, the MCU transmitted the heart rate and SpO₂ data via Bluetooth to a mobile device or cloud platform for further analysis. The device system was powered by a rechargeable lithium-ion battery, which could be wirelessly recharged with the help of bi-layered charging coils hot-pressed onto both sides of the TPU layer, which is separated from the lower circuit by a spacer. The laser-cut wireless charging coil in the serpentine shape also exhibited robust performance upon stretching, bending, and twisting (FIG. 12), as confirmed by the FEA using Ansys (Workbench 2020 R1). After soldering the battery to the FPCB, connecting the flexible sensors via flexible printed circuit connectors yielded the assembled system (FIG. 13).

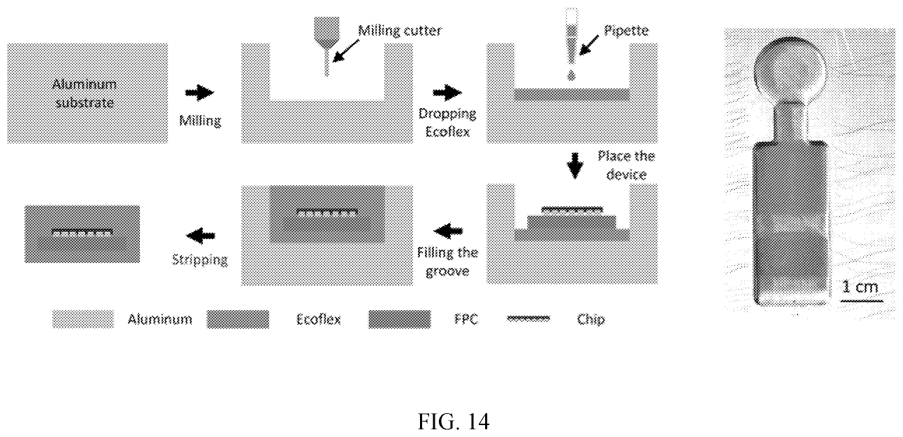

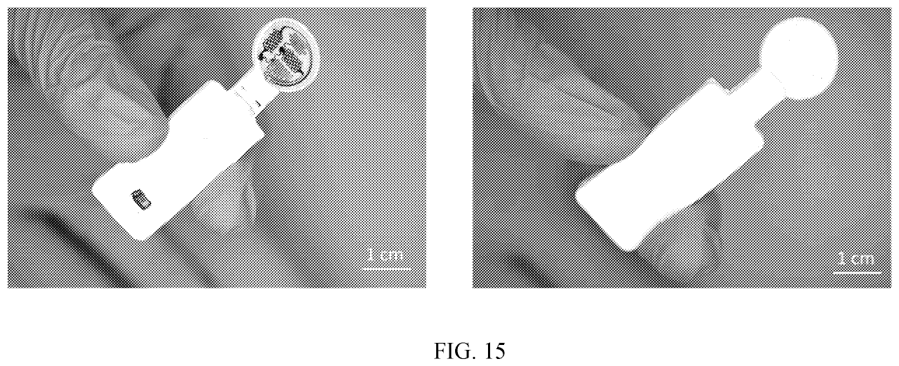

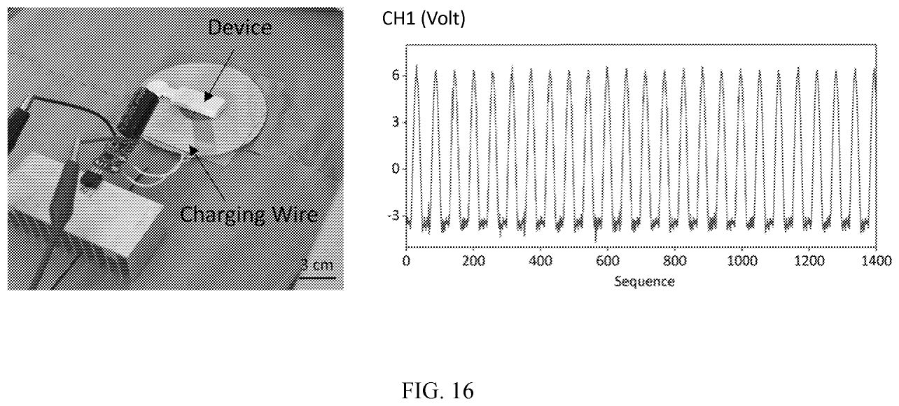

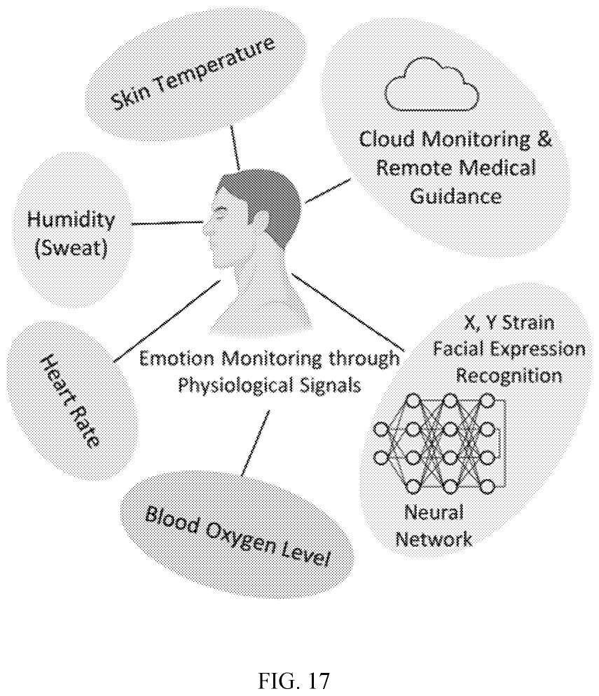

The assembled device is further encapsulated with Ecoflex (FIG. 14). The wireless charging module was also designed to operate in the presence of the encapsulation layer. Placing the encapsulated device system (FIG. 15) on the charging coil initiated the charging process. The AC voltage signal at the receiving coil (FIG. 16) was converted into a direct current (DC) voltage through unidirectional conduction and filtering, which was then used to charge the rechargeable lithium-ion battery in the FPCB for extended operation time of the device. As a result, the multimodal device sensing system could be deployed on the skin surface to monitor real-time vital physiological parameters over time for emotion detection (FIG. 17). As each emotion state can be associated with a distinct set of changes in the measured time-sequence physiological parameters, these valuable data processed by neural network-based classification learning allow for the recognition of facial expressions. Besides the intuitive display on the mobile interface, cloud platforms can be used to analyze data from large populations, enabling healthcare professionals to provide personalized emotional support for individuals with psychological conditions.

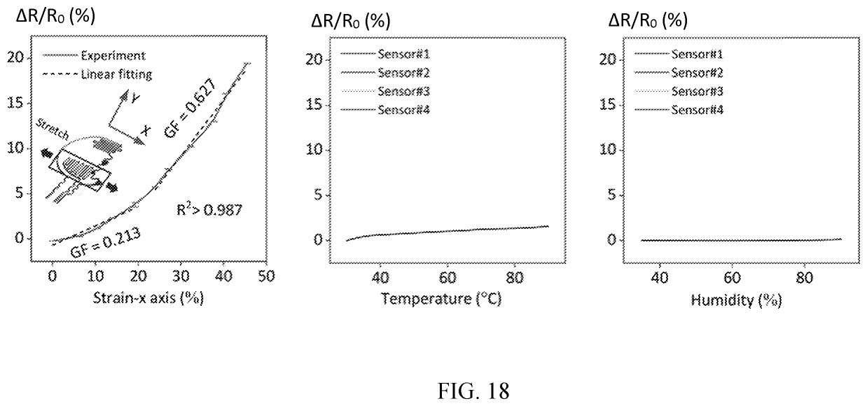

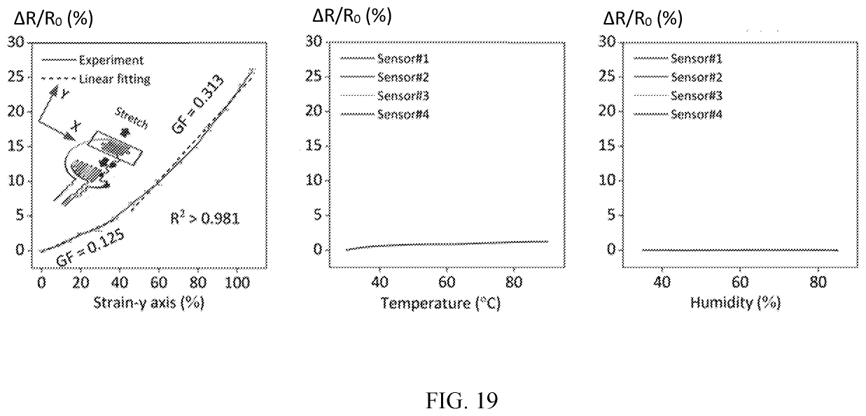

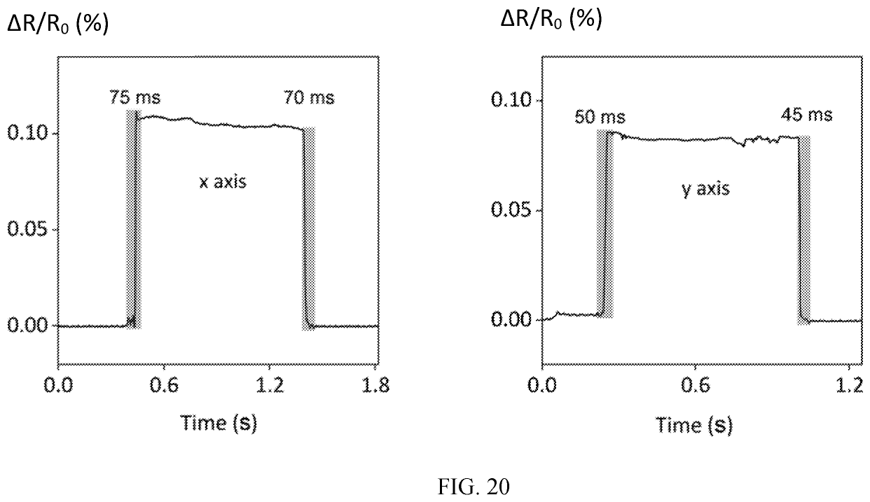

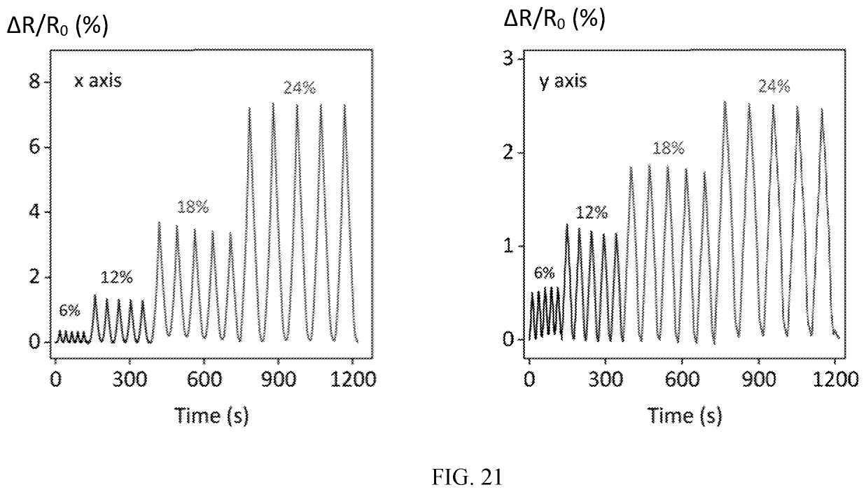

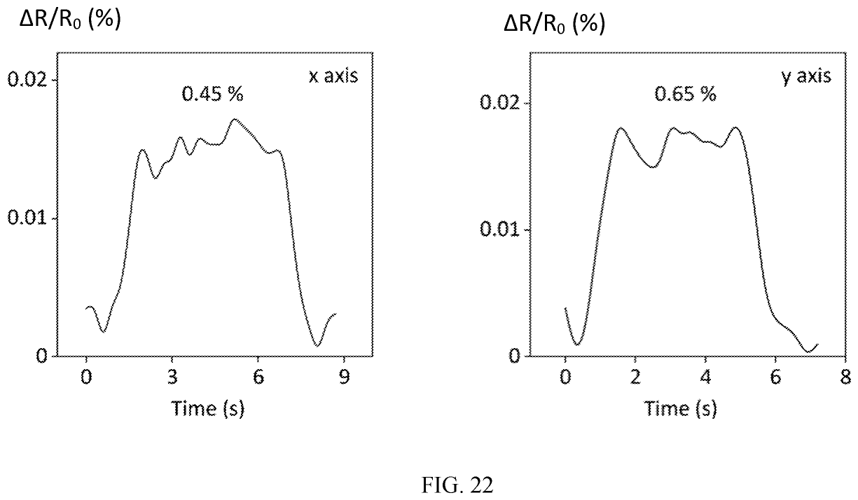

The Au-based strain sensor was piecewise linear in the strain range from 0 to 45% (or 0-100%) along the X-(or Y-) direction, with the sensitivity or gauge factor (GF) comparable with others in the literature or commercial products (FIGS. 18 and 19). More importantly, the strain sensors exhibited negligibly small changes to the temperature and humidity even over a wide range (FIGS. 18 and 19), indicating enhanced accuracy of the strain sensor in a complex environment with multiple input signals. Meanwhile, rapid response and recovery of 75 ms and 70 ms (or 50 ms and 45 ms) were observed in the X-axis (or Y-axis) strain sensor (FIG. 20). Besides highly stable and repeatable responses to dynamic strains of 6%, 12%, 18%, and 24% (FIG. 21), the sensor could also detect strains as small as 0.45% (or 0.65%) along the X- (or Y-) direction (FIG. 22).

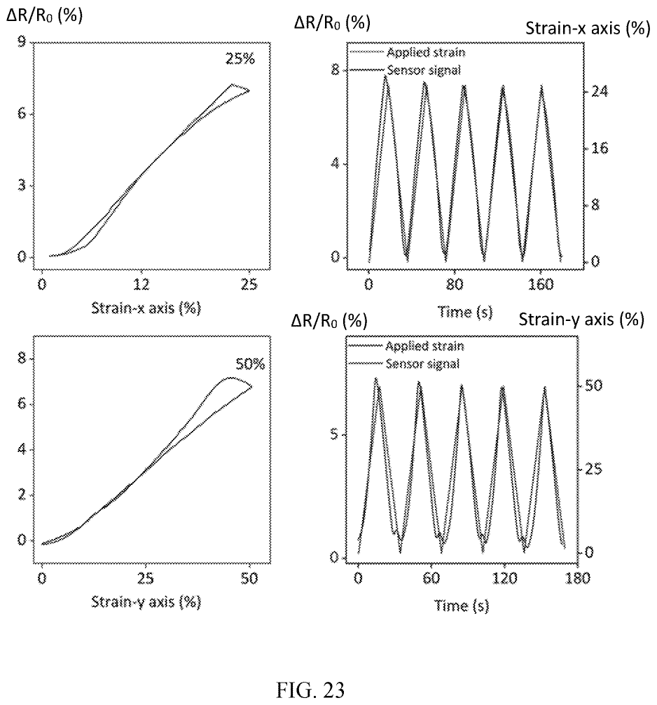

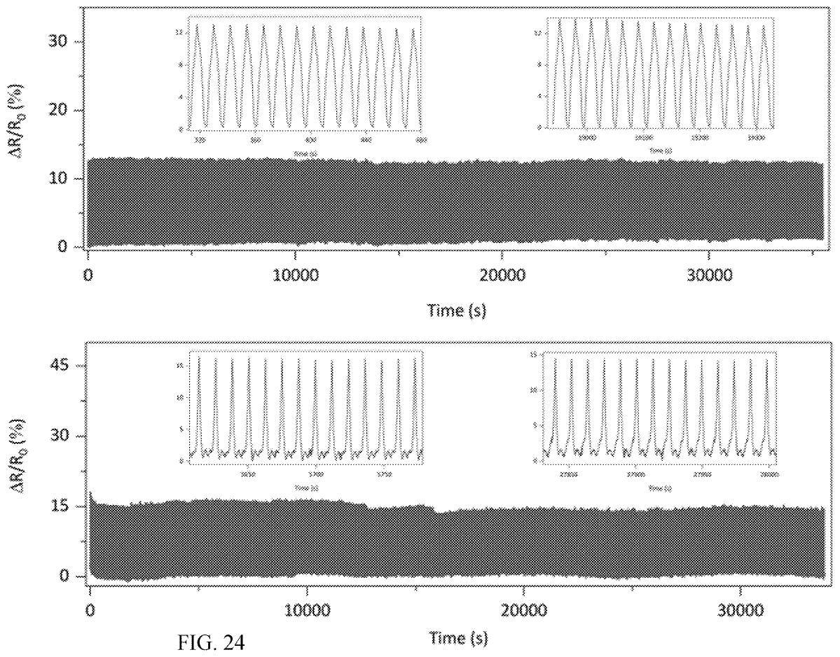

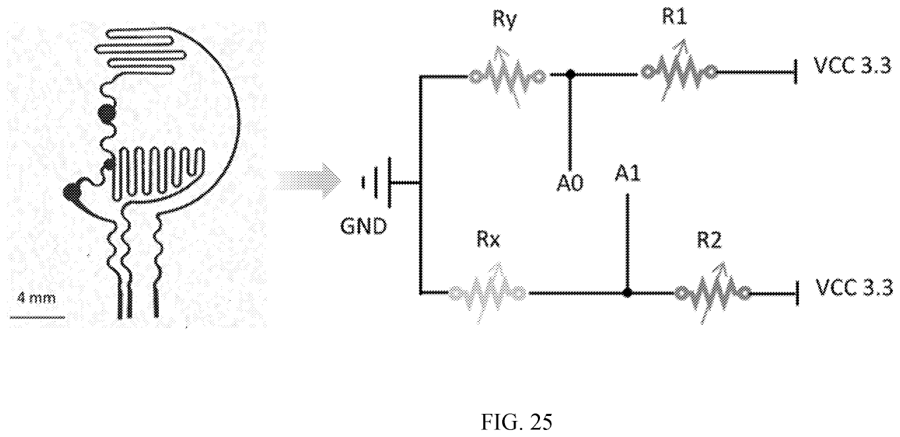

Despite the use of viscoelastic materials and complex interlayer contact conditions, a negligibly small delay occurred in the strain sensor between the strain and its response (FIG. 23). Cyclic stretching of the sensor at 35% (or 80%) strain in the X- (or Y-) direction over 10 hours also confirmed the excellent durability (FIG. 24). The circuit design with a common ground for the strain data readout (FIG. 25) ensured minimal interference between the X- and Y-axis sensors.

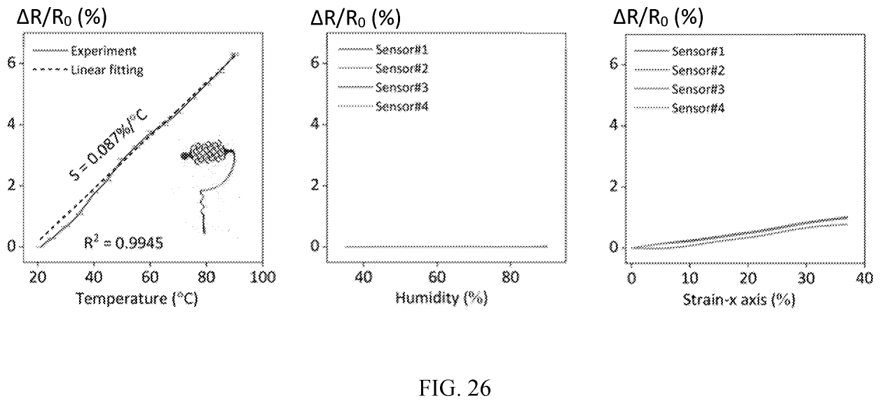

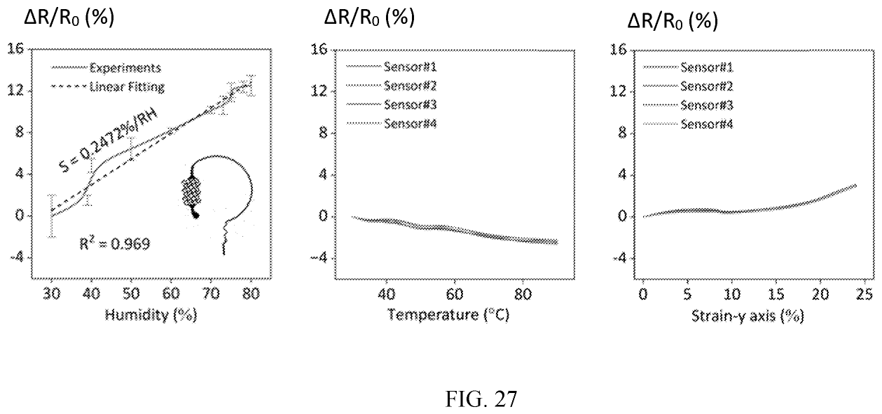

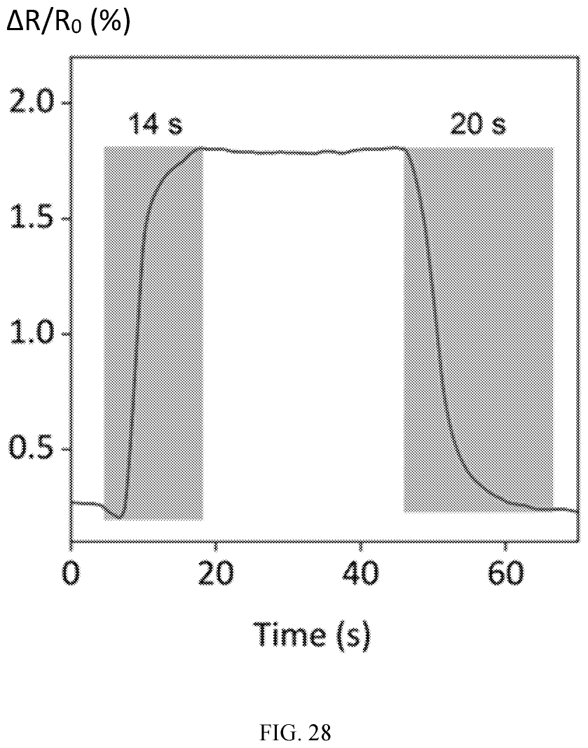

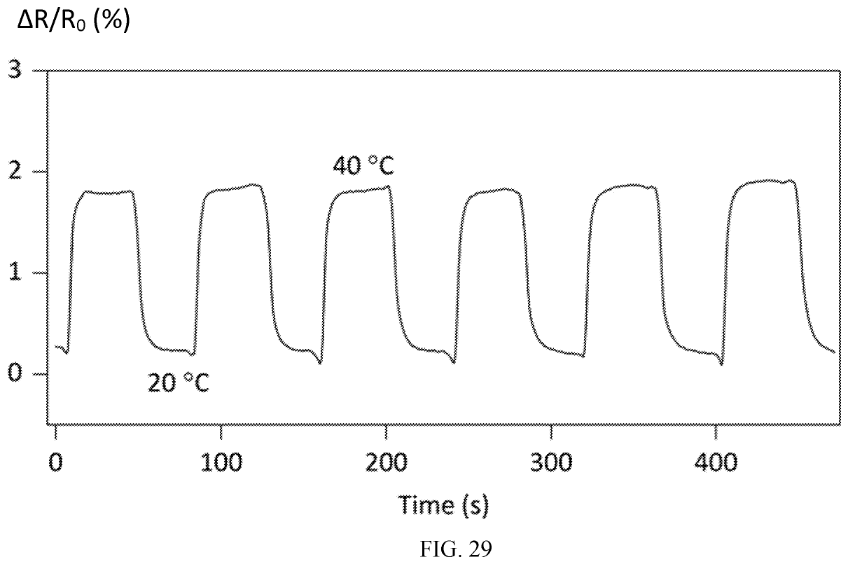

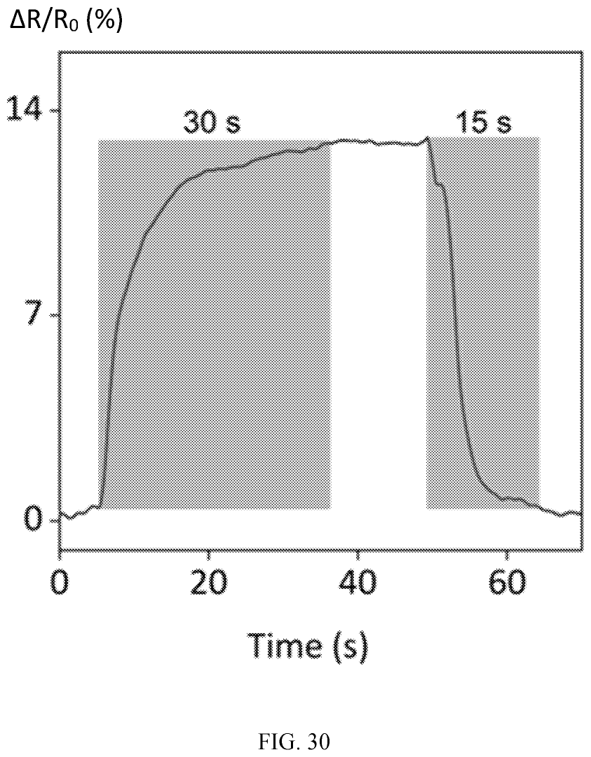

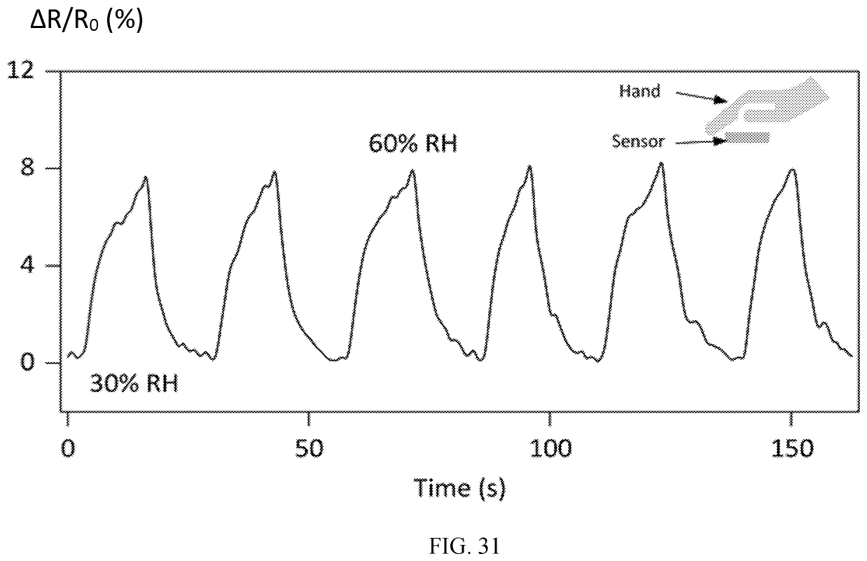

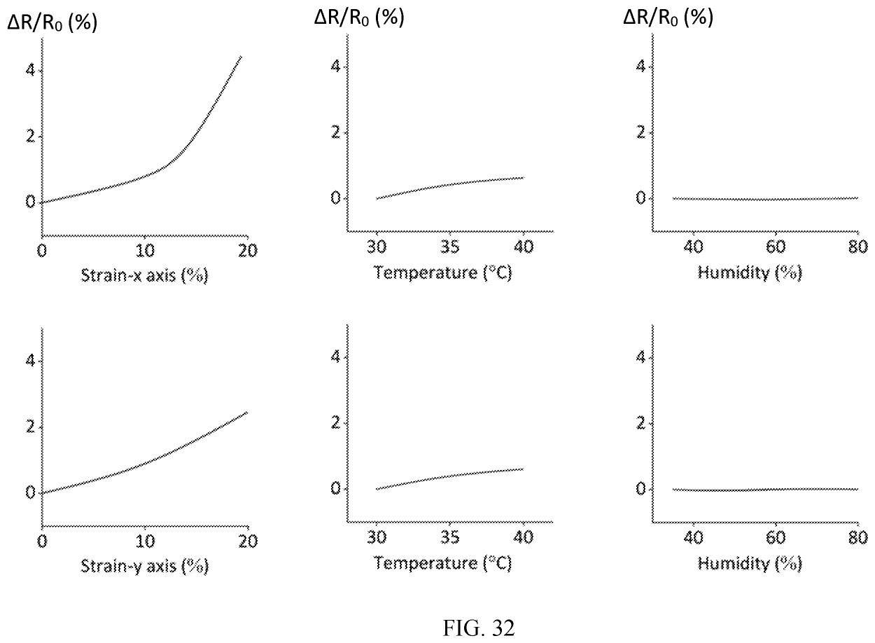

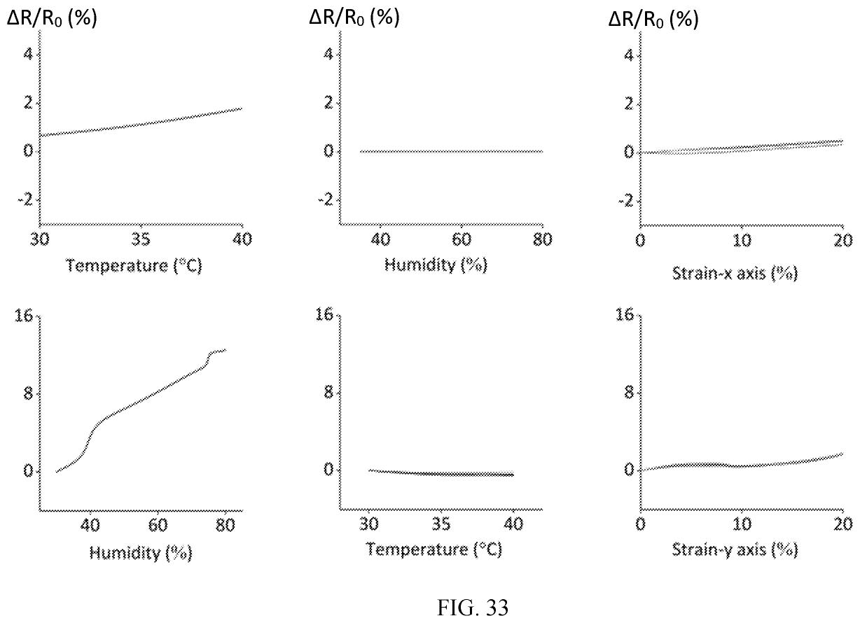

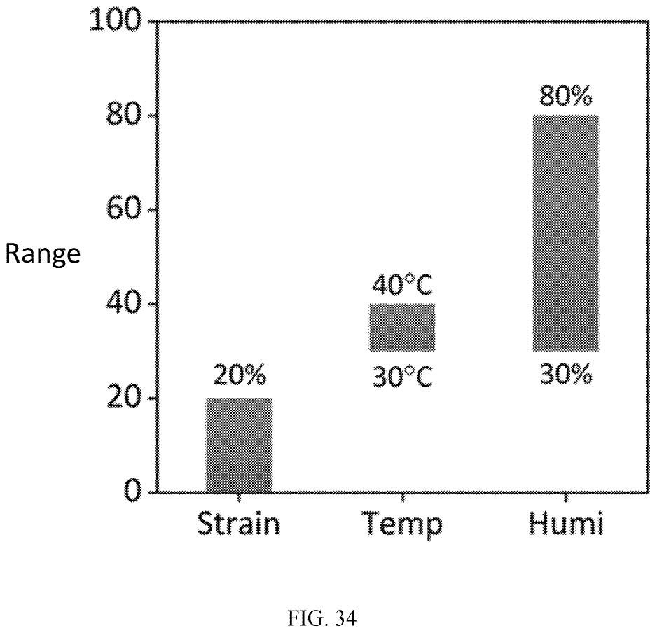

Relying on a positive temperature coefficient of resistance, the temperature sensor with a sensitivity of 0.087%/° C. in the linear range from 20 to 90° C. remained almost unaffected by the changes in the relative humidity due to isolation provided by the TPU, along with minimal impact by the strain (FIG. 26). Similarly, the humidity sensor with a sensitivity of 0.2472%/RH within the linear range from 30 to 80% RH exhibited lower sensitivity to temperature and strain (FIG. 27). Considering the encapsulation that impedes the heat transfer, the temperature sensor still showed a reasonably fast response/recovery time of 14/20 s, when cycled between 20° C. and 40° C. (FIG. 28). The continuous temperature changes over cycles could be stably measured (FIG. 29). Meanwhile, the humidity sensor with a response/recovery time of 30/15 s (between 30% and 60% RH) (FIG. 30) could effectively detect humidity changes, such as those caused by the approaching hand (FIG. 31). It should be noted that cross-interference among these sensors is further minimized (FIGS. 32 and 33), over the physiologically relevant ranges (FIG. 34) for the strain (0-20%), temperature (30-40° C.), and RH (30%-80%) on the human skin.

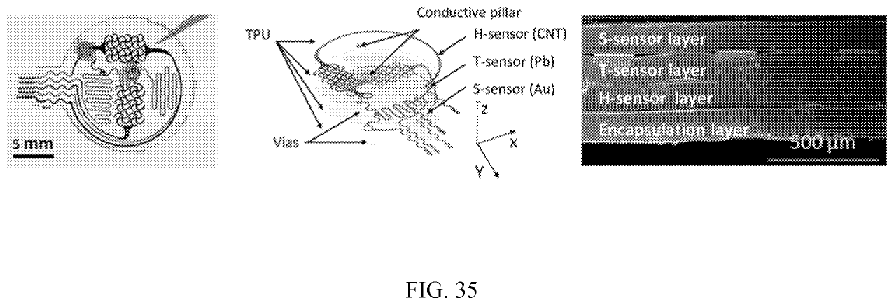

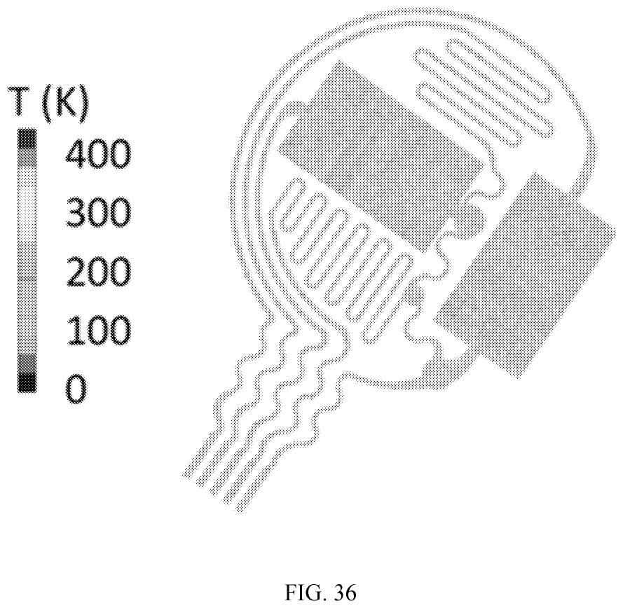

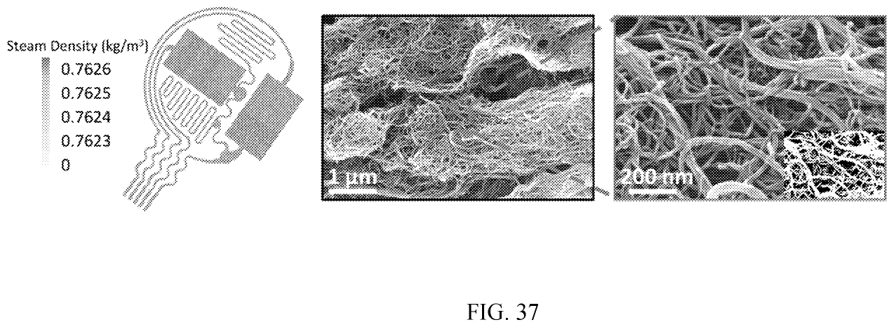

The encapsulated sensors arranged in a multi-layer layout were still compact due to the small thickness of each layer (FIG. 35). The simulation of the temperature field showed uniform distribution for efficient thermal conduction over the entire structure (FIG. 36). The absorption of water molecules onto the humidity sensor relied on the porous CNT with a porosity of approximately 37% (FIG. 37), disrupting the conductive pathways to increase the resistance. The resistance decrease in the CNT from increased temperature was markedly lower than that caused by the humidity, and the temperature determined by the temperature sensor could also be used to calibrate the humidity sensor. Additionally, it was possible to exploit porous materials that are insensitive to temperature for improved humidity-sensing performance.

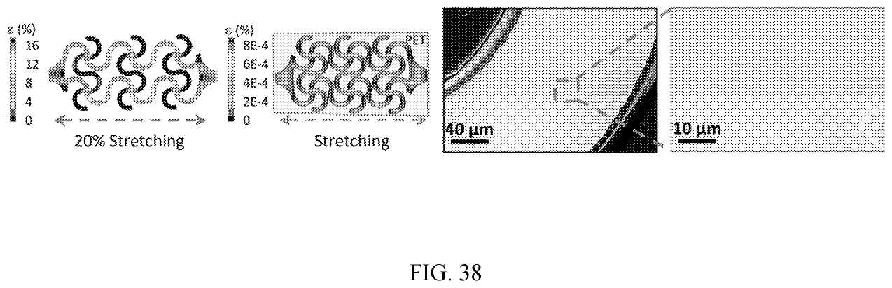

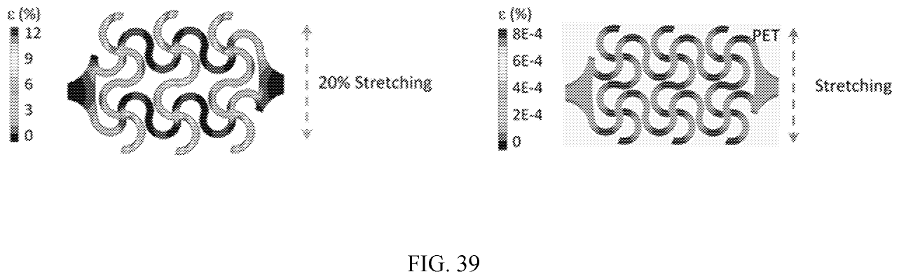

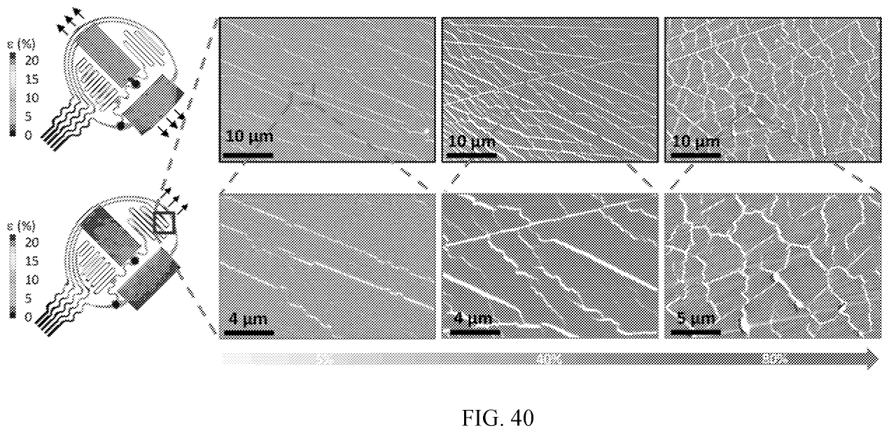

The temperature and humidity sensors were designed to be less sensitive to strain, as the stretchable serpentine structure reduced strain concentration during stretching (FIGS. 38 and 39). Combined with strain isolation provided by a high-modulus PET placed beneath, the effect of strain was further minimized (FIGS. 38 and 39), avoiding the generation of cracks in the serpentine (FIG. 38). However, strain concentration in the interconnect regions contributed to the stretching effect on the temperature and humidity sensors, which could be further reduced by increasing the rounding radius in these regions or selecting materials with strain insensitivity. Cracks mainly occurred in the serpentine of the strain sensor due to stress concentration during stretching along the X-(or Y-) direction (FIG. 40). The cracks formed along the perpendicular direction upon small stretching evolved into a mesh-like crack structure, accounting for the enhanced sensitivity with the increasing tensile strain. Meanwhile, the crack-induced resistance increase was much larger than that from temperature due to the thermal stability of gold, minimizing the effect of temperature on the strain sensor.

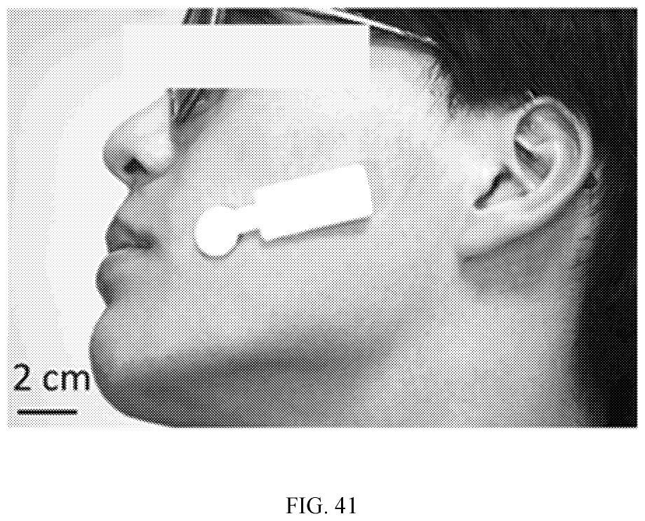

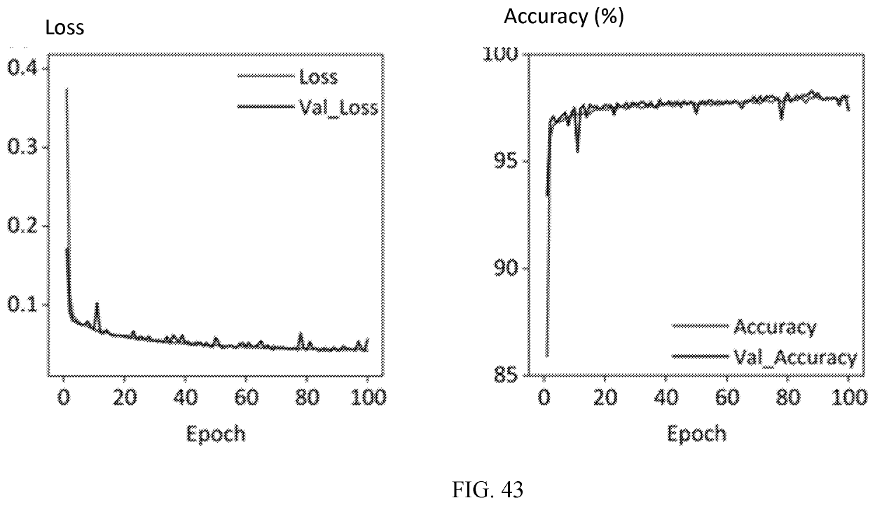

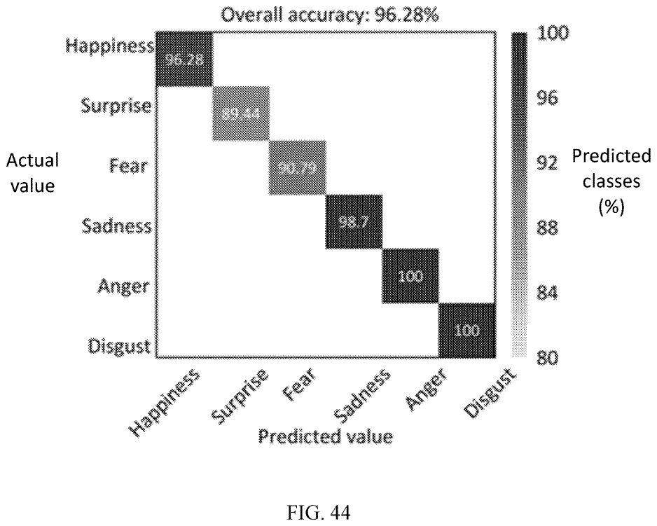

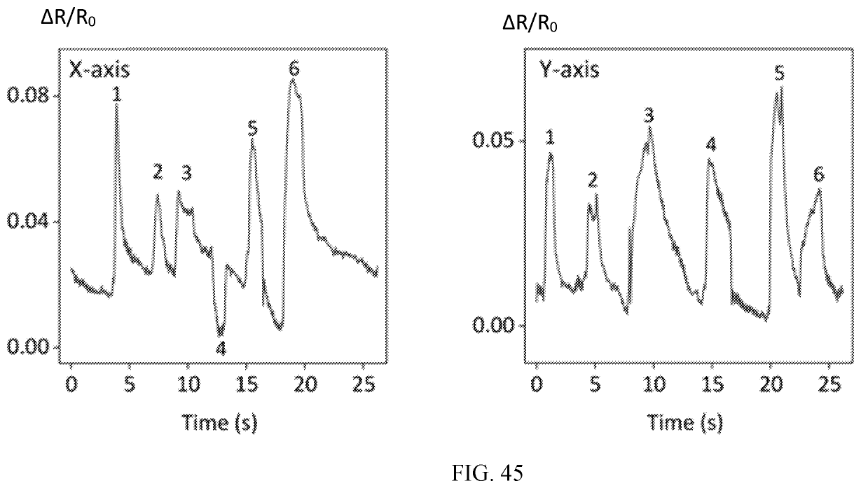

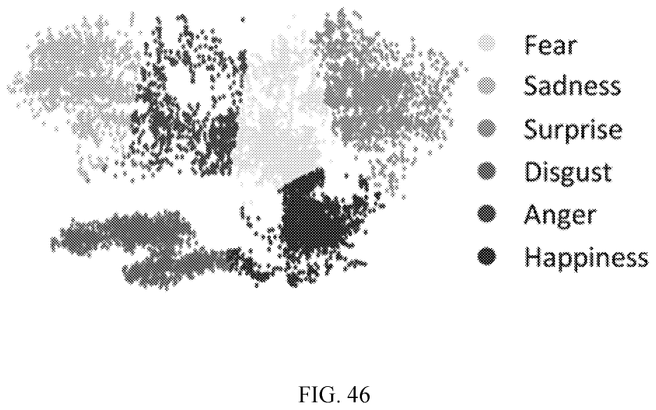

The multimodal sensors in the integrated hybrid electronics, to independently measure strain, temperature, humidity, heart rate, and SpO₂ with minimized interference and improved accuracy, provide opportunities to monitor emotions for understanding psychological conditions such as anxiety and panic disorders. As facial expression is the most direct manifestation of emotions, the integrated device system was attached to the face (FIG. 41). The collected 2D facial strain data could be processed by machine learning algorithms to classify common facial expressions (FIG. 42). With five human subjects repeating six facial expressions 100 times each, a reasonably large data set was established for training and validation of the neural network model. Over the 100 iterations or epochs during training, the model was continuously refined and iteratively optimized to improve classification accuracy. As a result, both training loss and validation loss decreased with the increasing iteration, which led to increased accuracy in both the training and validation data sets (FIG. 43). The optimized model achieved an overall accuracy of 96.28% to accurately classify six different facial expressions, as revealed by the confusion matrix (FIG. 44). The high recognition accuracy stems from the distinct sensor responses to each facial expression, with labels from 1 to 6 corresponding to happiness, surprise, fear, sadness, anger, and disgust, as highlighted in the representative data from one of the human subjects (FIG. 45). The clear separations between the features of different facial expressions in the 2D K-means clustering results also confirm the distinct groupings (FIG. 46).

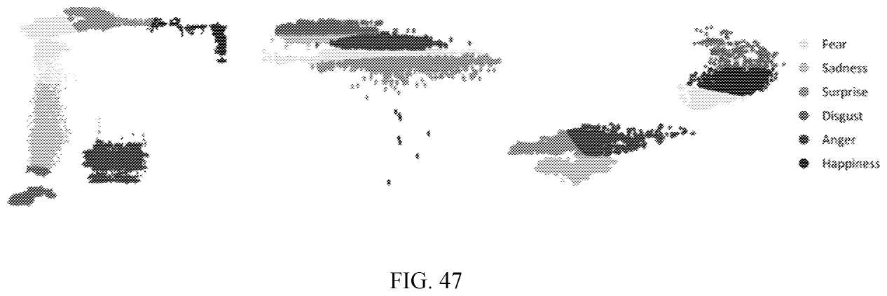

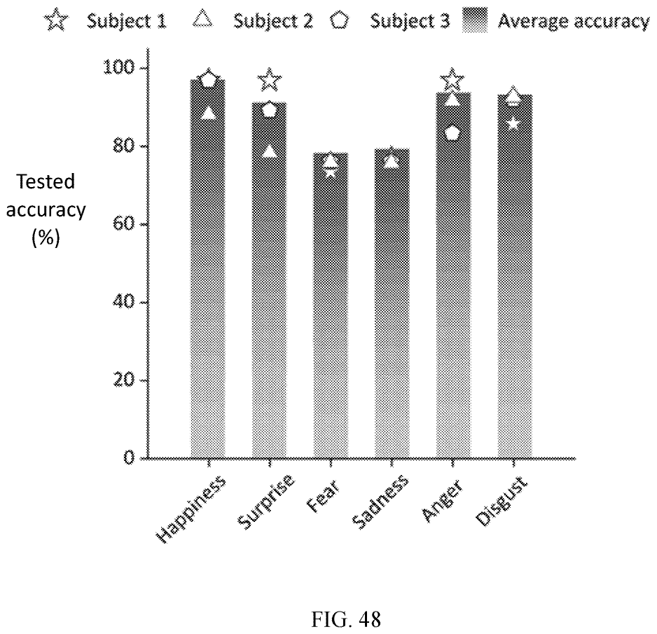

The model featured a robust performance of emotion recognition with facial expression data collected from three new human subjects. Despite variations in facial expressions among different individuals, the 2D K-means clustering results still demonstrated clear distinctions between the features of facial expressions within each subject (FIG. 47). The classification accuracy with an average of 88.83% (FIG. 48) confirmed that the model adapts well to individual differences.

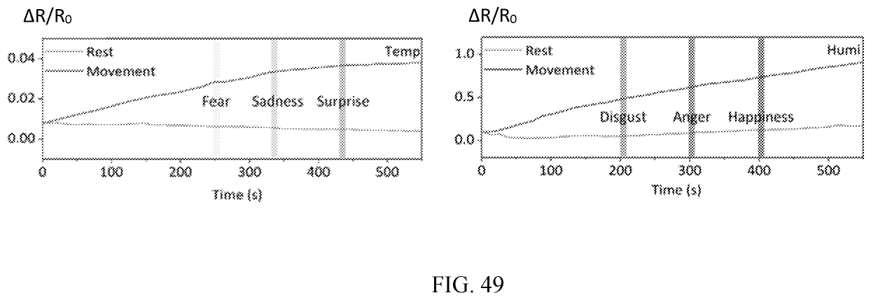

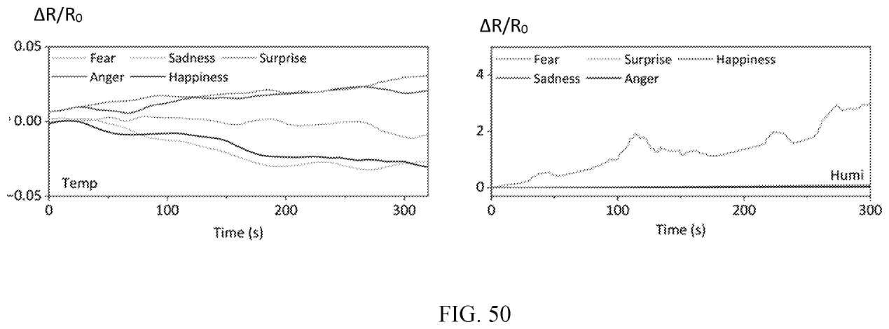

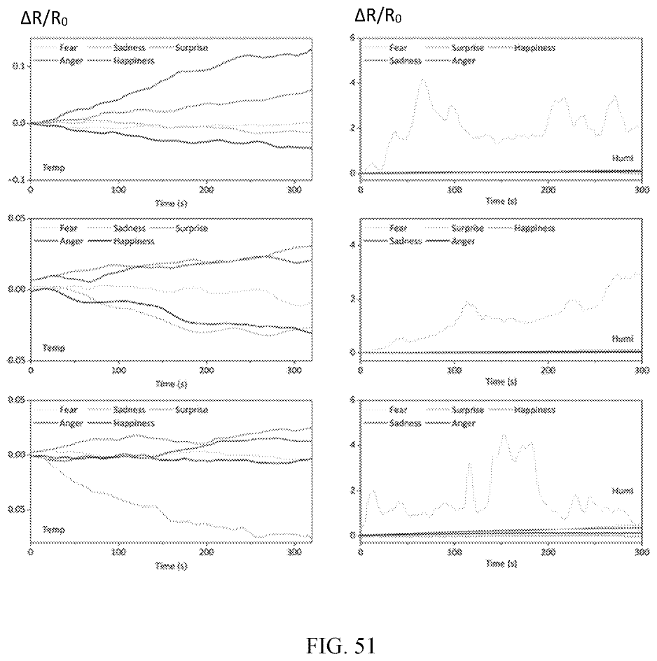

Although facial expressions can intuitively display emotions, individual differences and social factors can render subtle or even invisible expressions of many emotions on the face. Therefore, it is highly desirable to combine changes in skin temperature, humidity, heart rate, and blood oxygen levels to provide a more comprehensive assessment and recognition. To test the hypothesis, the temperature and humidity data were first collected from the device applied to the face in both resting and moving states (FIG. 49). Compared with the resting state, the movement-induced rise in temperature and humidity was captured by both sensors. The simulated facial expressions corresponding to the emotion but without actual emotional changes were introduced at approximately 250 s (fear), 350 s (sadness), and 450 s (surprise) during the temperature measurement, which exhibited a negligibly small influence. Similarly, the simulated disgusted, angry, and happy expressions introduced at around 200 s, 300 s, and 400 s did not show effects on the measured humidity. In comparison, body temperature and humidity were affected by emotional responses or changes elicited by watching video clips. For instance, skin temperature was slightly increased during surprise and anger, but decreased during fear, sadness, and happiness (FIG. 50). Meanwhile, the increased skin humidity was solely observed in fear, due to perspiration (FIG. 50). It should be noted that emotion-induced changes in temperature and humidity showed high individual differences or even differ at different times of the day. Fortunately, the overall trends remained consistent throughout the day, despite the differences in the response magnitude (FIG. 51), which suggests their potential use for robust performance.

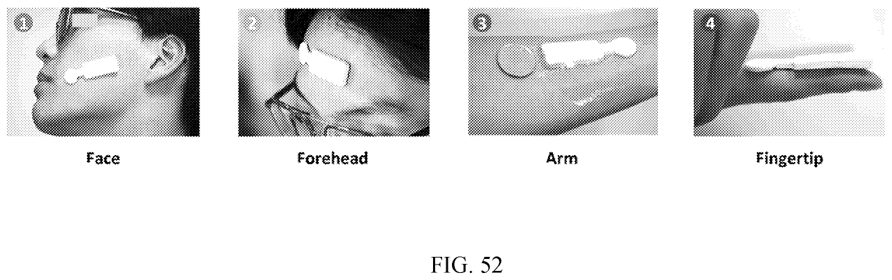

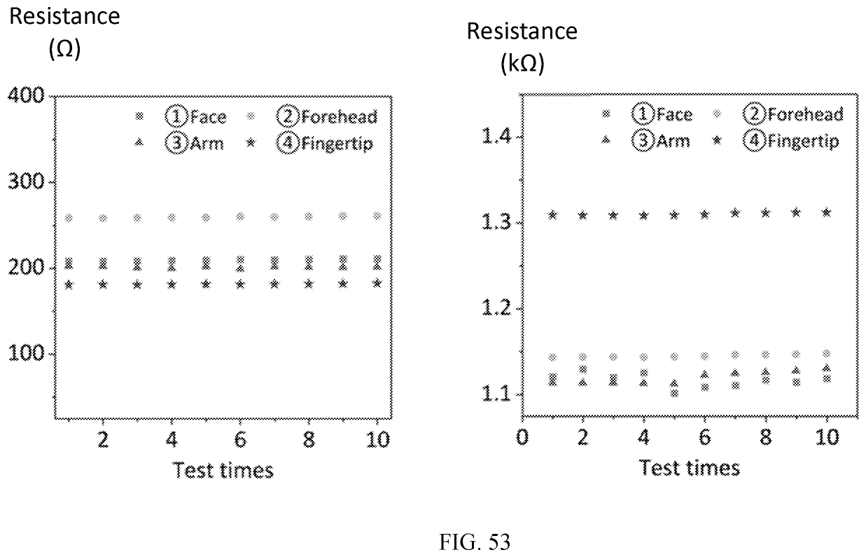

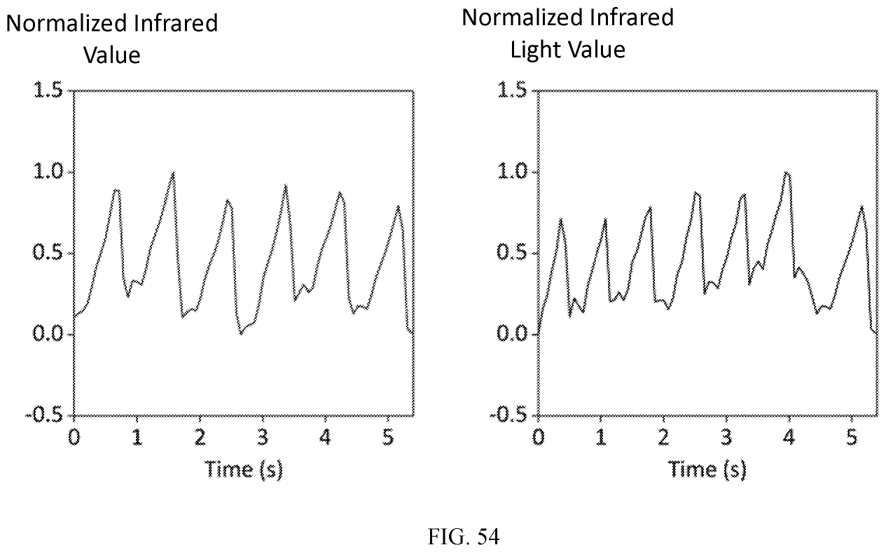

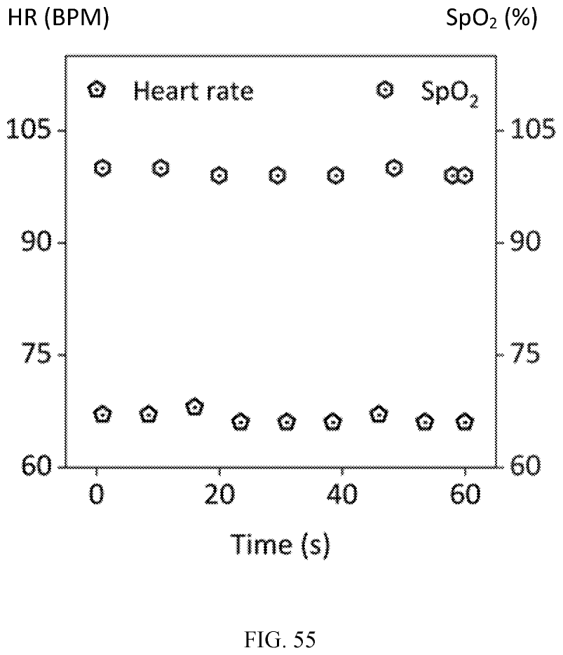

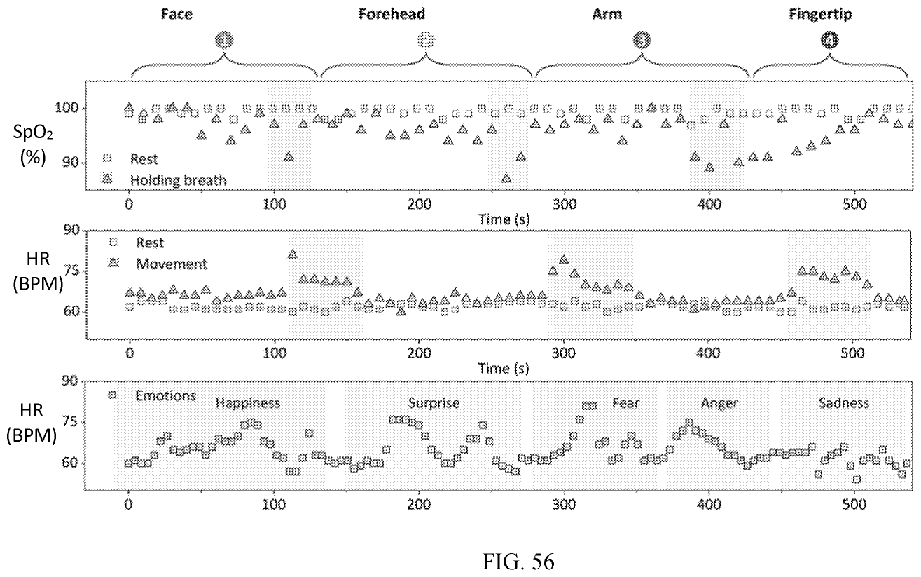

The integrated hybrid device could also be applied to different body parts (e.g., the face, forehead, arm, and fingertip) (FIG. 52) to quantify the temperature and humidity distribution across the body (FIG. 53). The raw normalized PPG voltage output was processed by the MCU to calculate heart rate and SpO₂ signals (FIG. 54). Motion artifact in the PPG signals was identified and filtered out by the MCU algorithm, which ensured data accuracy in the measured heart rate and SpO2 even in the presence of approximately 10% dynamic stretching on the forearm (FIG. 55). The accurate monitoring of reduced SpO₂ can be particularly relevant to detecting dyspnea that results from prolonged anxiety or panic. The PPG sensor can measure SpO₂ value in resting and intermittent breath-holding states from different body parts (FIG. 56).

Besides collecting the heart rate at rest and during movement (FIG. 56), the changes in the heart rate during various emotional states evoked from watching video clips can also be captured (FIG. 56). These results, collectively, demonstrated that the additional measurements of skin temperature, humidity, and heart rate can help rule out fake facial expressions for detecting true emotions.

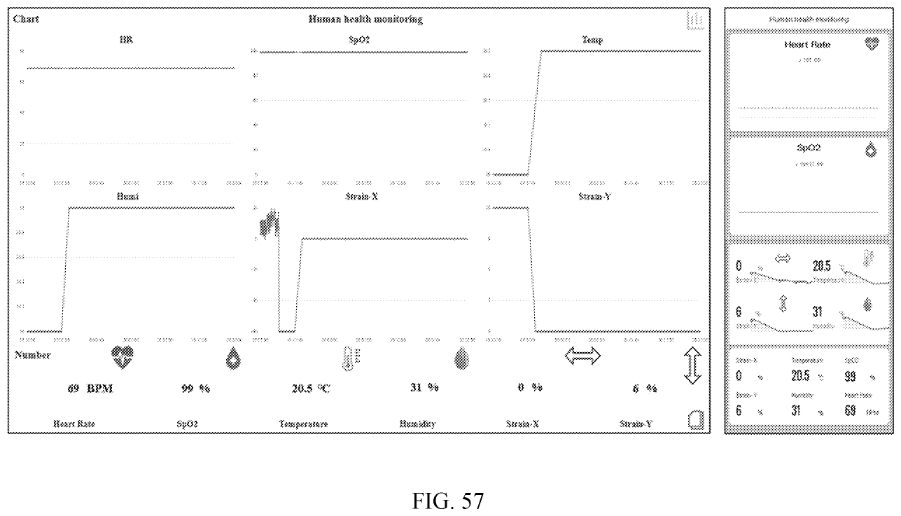

Transmitting real-time data to a mobile device and uploading historical data to the cloud (FIG. 57) further allowed the integrated system to continuously monitor individualized emotions across a large population, providing emotional support from healthcare professionals through telemedicine when necessary.

In summary, this example introduced a fully integrated multimodal stretchable hybrid electronic device for emotion monitoring. Through optimized structural design and material selection, multimodal sensors could effectively minimize coupling and interference to accurately detect strain, temperature, and humidity in real-time, along with heart rate and SpO₂ measured by the PPG sensor. Both the sensors and FPCB can be vertically integrated into a double-layer layout for a minimized device footprint. A neural network model trained using temperature- and humidity-independent facial strain data featured high-precision facial expression recognition. The emotion-relevant physiological signals, such as temperature and humidity, along with heart rate and SpO₂, were also validated to provide comprehensive data linked to emotions, presenting a promising future research direction. By transmitting the measured multimodal data to mobile devices and clouds, the system can allow healthcare professionals to analyze psychological health and provide timely emotional intervention.

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the endpoints. Thus, while certain exemplary embodiments of the device and methods of making and using the same have been discussed and illustrated herein, it is to be distinctly understood that the invention is not limited thereto but may be otherwise variously embodied and practiced within the scope of the following claims.

What is claimed is:

1. A sensing device comprising:
a flexible printed circuit board comprising:
a microcontroller unit,
a non-transitory computer-readable medium connected to the microcontroller unit,
a Bluetooth transceiver unit connected to the microcontroller, and
a sensor array connected to the microcontroller,
wherein the sensor array comprises:
a biaxial strain sensor configured to collect skin strain data,
a temperature sensor configured to collect skin or environmental temperature data,
a humidity sensor configured to collect skin moisture data, and
a photoplethysmography sensor configured to collect heart rate data and blood oxygen saturation data.

2. The sensing device of claim 1, wherein the sensing device comprises an inside surface configured to contact a user when in use, and an outside surface opposite the inside surface, and
wherein the sensor array is vertically stacked.

3. The sensing device of claim 2, wherein the sensor array is vertically stacked such that the humidity sensor is proximal to the inner surface.

4. The sensing device of claim 3, wherein the humidity sensor comprises a hydrophobic layer with an opening to enable the humidity sensor to be exposed while protecting other sensors from moisture.

5. The sensing device of claim 3, wherein the temperature sensor comprises a hydrophobic layer to protect the temperature sensor from moisture.

6. The sensing device of claim 2, wherein a rigid layer is positioned adjacent to the humidity sensor to minimize the effects of strain.

7. The sensing device of claim 2, wherein a rigid layer is positioned adjacent to the temperature sensor to minimize the effects of strain.

8. The sensing device of claim 1, wherein the sensor array is positioned such that the collected skin strain data, skin or environmental temperature data, skin moisture data, heart rate data, and blood oxygen saturation data are decoupled.

9. The sensing device of claim 1, wherein the device exhibits interference-resistant performance, with a normalized relative resistance change (ΔR/R₀) of 2% or less over an x-strain range of 20% or less, a y-strain range of 20% or less, a temperature range of 30-40 C., and a relative humidity range of 30-80%.

10. The sensing device of claim 1, wherein the biaxial strain sensor comprises an x-axis strain sensor and a y-axis strain sensor.

11. The sensing device of claim 1, wherein at least one of the biaxial strain sensor, the temperature sensor, the humidity sensor, and the photoplethysmography sensor are designed in a serpentine shape.

12. The sensing device of claim 1, wherein each of the biaxial strain sensor, the temperature sensor, and the humidity sensor are designed in a serpentine shape.

13. The sensing device of claim 1, further comprising a power source configured to facilitate wireless charging and recharging of the sensing device.

14. The sensing device of claim 13, wherein the power source comprises one or more charging coils designed in a serpentine shape.

15. A method for detecting emotions, the method comprising:
providing the sensing device of claim 1;
collecting sensor data via the sensor array; and
transmitting the collected sensor data to a machine learning model.

16. The method of claim 15, further comprising:
processing the collected sensor data via the machine learning model to detect emotions.

17. The method of claim 15, wherein the machine learning model is hosted on an input/output device or a server.
